(19) United States
(12) Patent Application Publication
(10) Pub. No.: US 2026/0151370 A1
(43) Pub. Date: Jun. 4, 2026

(54) Nutritional composition for neurodegeneration and neurotrauma

(71) Applicant: N.V. Nutricia, Zoetermeer (NL)

(72) Inventors: Ladislaus Maria Broersen, Utrecht (NL); Lisanne Marijn Roovers, Utrecht (NL)

(73) Assignee: N.V. Nutricia, Zoetermeer (NL)

(21) Appl. No.: 19/427,051

(22) Filed: Dec. 19, 2025

Related U.S. Application Data
(63) Continuation of application No. PCT/EP2024/068166, filed on Jun. 27, 2024.

(30) Foreign Application Priority Data
Jun. 30, 2023 (EP) 23182879.9
Dec. 27, 2023 (EP) 23220387.7

(51) Int. Cl. A61K 31/202; A61K 9/00; A61K 31/07; A61K 31/14; A61K 31/19; A61K 31/197; A61K 31/355; A61K 31/375; A61K 31/4188; A61K 31/4415; A61K 31/455; A61K 31/51; A61K 31/519; A61K 31/525; A61K 31/593; A61K 31/685; A61K 31/702; A61K 31/714; A61K 31/732; A61K 33/04; A61K 33/06; A61P 25/00

(52) U.S. Cl. CPC A61K 31/202; A61K 9/0053; A61K 9/0095; A61K 31/07; A61K 31/14; A61K 31/19; A61K 31/197; A61K 31/355; A61K 31/375; A61K 31/4188; A61K 31/4415; A61K 31/455; A61K 31/51; A61K 31/519; A61K 31/525; A61K 31/593; A61K 31/685; A61K 31/702; A61K 31/714; A61K 31/732; A61K 33/04; A61K 33/06; A61P 25/00

(57) ABSTRACT

The invention relates to a nutritional composition comprising a combination of (i) omega-3 fatty acid DHA with (ii) vitamin B2 and (iii) vitamin D3 and one or more from (iv) choline or a salt thereof and/or choline esters; and (v) dietary fibres and/or dietary butyrate for use in the treatment of neurodegenerative diseases and/or neurotrauma, brain injury and/or brain damage. The invention also relates to a composition comprising a combination of DHA, vitamin B2, vitamin D3 with one or more from (iv) choline or a salt thereof and/or choline esters; and (v) dietary fibres and/or dietary butyrate.

### FIELD OF THE INVENTION

The present invention is in the field of compositions and nutritional compositions for use in treating neurodegenerative diseases, preferably treating and/or improving the symptoms of neurodegenerative diseases and their use in neurotrauma/brain trauma and/or neurological damage caused by trauma and/or improving functional recovery after brain damage.

### BACKGROUND

Neurite outgrowth refers to the process by which nerve cells, also known as neurons, extend their projections called neurites. Neurites include both axons and dendrites. Axons transmit electrical or neurochemical signals to other neurons, while dendrites receive signals from neighbouring neurons.

When neurons undergo neurite outgrowth, it allows them to establish connections and communicate with other neurons in the brain. These connections are essential for the functioning of the brain and form what is known as the functional connectivity. Functional connectivity refers to the patterns of communication and interaction between different brain regions which involves the synchronized activity of multiple neurons and neural circuits. Neurite outgrowth plays a crucial role in shaping these connections and influencing how brain regions communicate with each other.

Neurons extend their neurites towards specific target areas and seek out other neurons or regions of the brain to form connections, allowing the establishment of neural circuits and enabling information processing and transmission. The growth of neurites helps in the formation of synapses, which are the points of connection between neurons, thereby contributing to the overall functional connectivity of the brain. The complex network of neurons is responsible for various brain functions, including sensory perception, motor control, sleep, memory, and cognitive processes by means of sending and receiving signals.

Neurodegenerative diseases can disrupt the functional connectivity of the brain, primarily through the accumulation of abnormal proteins, inflammation, and loss of supportive glial cells. Impaired neurite outgrowth in neurodegenerative diseases affects the ability of neurons to form new connections and maintain functional connectivity in the brain. Together this leads to the progressive decline in brain function and the characteristic symptoms associated with these diseases, such as memory loss, movement difficulties, sleep disturbances, and cognitive impairment.

Treatment of neurodegenerative diseases focuses on medication, delaying the disease progress, physical therapy, lifestyle modifications and supportive care. Regarding lifestyle modifications, a balanced diet has been shown to play a role in disease management and to slow down disease progression.

CN107889992 describes a functional health beverage for preventing or treating dementia containing omega-3 fatty acids, choline, and uridine as active ingredients to reduce disease progression and further comprising vitamins and trace elements. WO2021/034942 describes a dietary supplement ameliorating dietary inadequacies related to brain health or neurodegenerative disease containing two polyphenolic compounds, an omega-3 fatty acid, a water-soluble and oil-soluble vitamin component and magnesium. CN112890136 describes an antioxidant total nutrition powder as adjuvant therapy for the treatment of Alzheimer's disease comprising DHA, B vitamins, vitamin D, phospholipids, and taurine among many other ingredients. WO2018/226089 focuses on treating impaired motor skills in mammals suffering from Parkinson's disease (PD) and mentions presence of omega-3 fatty acids, choline and D3 and refers to dietary fibres for its neuroprotective effect in PD patients.

Neurotrauma refers to an injury or damage to the brain, spinal cord, or peripheral nerves, typically resulting from a sudden traumatic event. It encompasses a broad range of injuries, including traumatic brain injury (TBI), spinal cord injury (SCI), and peripheral nerve injuries. Traumatic brain injury (TBI) occurs when there is a sudden blow, jolt, or penetrating injury to the head, leading to disruption of normal brain function. TBIs can range from mild concussions to severe injuries causing long-term or permanent neurological deficits. SCI refers to damage to the spinal cord, resulting in loss of motor and sensory function below the level of injury. SCI can lead to varying degrees of paralysis, depending on the location and severity of the injury. Peripheral nerve injuries involve damage to the nerves located outside the brain and spinal cord. These injuries can occur due to trauma, such as lacerations, fractures, or compression, and can result in loss of sensation or motor function in the affected area. Neurotrauma can have significant consequences and may result in physical, cognitive, and psychological impairments. In this regard, neurotrauma can have both direct and indirect effects on neurite outgrowth, depending on the severity and type of injury.

Several ways by which neurotrauma can impact neurites are: disruption/disconnection of neuronal structure, including axons and dendrites; axonal degeneration resulting in the retraction or loss of axonal processes; excessive or chronic inflammation can inhibit neurite outgrowth; scar tissue formation at the injury site can create physical barriers that impede the extension of neuronal processes; altered cellular environment via release of inflammatory molecules, excitotoxicity, and changes in the balance of neurotransmitters impacting neurite outgrowth by influencing the availability of growth factors and guidance cues necessary for proper neuronal development and regeneration. However, in some cases, neuronal regeneration can occur, with regrowth of damaged or severed axons. The extent and success of regeneration depend on various factors, including age, overall health, the severity of the injury, the presence of inhibitory factors in the environment, and the capacity of the injured neurons to regrow.

WO2012125034 describes a composition comprising: i) one or more of uridine and cytidine, or salts, phosphates, acyl derivatives or esters thereof; ii) a lipid fraction comprising at least one of docosahexaenoic acid (22:6; DHA), eicosapentaenoic acid (20:5; EPA) and docosapentaenoic acid (22:5; DPA), or esters thereof, in which the lipid fraction comprises less than 2 weight % of α-linolenic acid (ALA), based on the weight of all fatty acids; iii) choline, or salts or esters thereof; for use in the prevention or treatment of neurotrauma, TBI, cerebral palsy and SCI.

WO2019013615 describes a composition for use in producing white matter, producing myelin and/or reducing brain lesion size in a mammal suffering from or recovering from traumatic brain injury, comprising enterally administering to the subject a composition comprising therapeutically effective amounts of: (i) one or more of uridine, cytidine, or salts, phosphates, acyl derivatives or esters thereof; (ii) a lipid fraction comprising at least one of DHA, EPA and DPA, wherein the lipid fraction comprises less than 2 weight % of ALA, calculated on the weight of all fatty acids; (iii) choline, or salts or esters thereof; and (iv) at least one vitamin B selected from vitamins B6, B9 and B12. US20170127693 describes a nutritional composition comprising butyrate, DHA, phosphatidylcholine, recommended daily amounts vitamin D and B2 for paediatric use, but is silent on the addition of dietary fibres or neurological applications of said composition.

WO2016014473 describes a composition comprising a human milk oligosaccharide (or precursor) and mentions a formulation further comprising DHA, vitamin B2 and vitamin D3 among other ingredients. It mentions a long list of uses, among which neurodegenerative diseases are mentioned but it is silent on neurotrauma or brain injury and furthermore silent on the presence of phosphatidylcholine.

WO2019165222 describes nutritional daily supplements for kidney diseases that contain vitamin B2 and D3 levels above recommended daily amounts (RDA). It is indicated that the supplements can be added together with omega 3 fatty acids. It is silent on choline and dietary fibres and does not mention neurotrauma and brain injury as a therapeutic application.

EP2342975 describes a composition containing omega-3 fatty acids, protein, manganese or molybdenum, vitamin B2, vitamin D3 and choline for treating Alzheimer's disease but it does not mention dietary butyrate or phosphatidylcholine. It is indicated that the vitamins B6, B9 and B12 may be present in amounts that exceed the RDA for humans but the amounts of vitamin B2 and D are only added in amounts that match the RDA.

Current dietary options include many different nutrients yet those have shown mixed results in different combinations of nutrients. So, an ongoing need remains to provide for find ways nutritional supplements or nutritional compositions to treat neurodegenerative diseases and neurotrauma.

### SUMMARY OF THE INVENTION

The inventors unexpectedly found that a composition comprising a combination of therapeutically effective amounts of the omega-3 fatty acid docosahexaenoic acid (DHA), vitamin B2 and vitamin D3 is effective in the treatment of neurodegenerative diseases, neurotrauma and brain injury/brain damage. Treatment of neurodegenerative diseases includes treatment of the symptoms of these neurodegenerative diseases and treatment of neurotrauma includes treatment of neurotrauma caused by external factors. Treatment of brain injury/brain damage includes improving neurological recovery after brain injury or damage has occurred.

Surprisingly a composition was developed that comprises a limited number of ingredients that work synergistically. This composition was found to reduce the risk of counteracting mechanisms of action in neurodegenerative diseases and neurotrauma and/or in brain injury/brain damage or reduce the risk of undesirable side effects to patients. Without being bound by theory, it is believed that the therapeutically effective amounts of the omega-3 fatty acid DHA, vitamin B2 and vitamin D3 in a synergistic manner reduce the effects of oxidative stress and inhibit inflammation, regulate synaptic plasticity and molecular transport in cell organelles.

It was found in an in vitro PC12 model that a composition comprising a combination of omega-3 fatty acid DHA, vitamin B2, and vitamin D3 results in an increase of neurite outgrowth (as defined by the number of neurites and/or neurite length per cell) as compared to a composition without these components. The increase in neurite outgrowth, referring to the number of neurites and/or neurite length per cell, in PC12 cells was higher than expected based on the additional effect of each individual component thereby showing a synergistic effect between these three components.

It was further observed that adding choline and in particular phosphatidylcholine (PC) to the combination of DHA, B2 and D3 further improved neurite outgrowth, also to a more than expected extend based on the effect of choline or PC individually, when added to the effect of DHA, B2 and D3. A similar more than additive effect was also observed when butyric acid was supplemented together with DHA, B2, and D3. In addition, it was found that supplementing PC12 cells with a combination of all five aforementioned components unexpectedly resulted in an even further improvement, i.e. increase, in neurite outgrowth compared to the effect of each individual component added together, thereby indicating synergy of these components on increasing neurite outgrowth.

The inventors have further surprisingly discovered that a combination of a small number of nutrients comprising omega-3 fatty acid DHA, vitamin B2 and vitamin D3 and optionally adding choline, in particular phosphatidylcholine (PC) and/or butyric acid to the combination has a synergistic effect on neurite outgrowth.

An increase in neurite outgrowth refers to the density of neurites reflecting the number of neurites and/or neurite length per cell, preferably the number of neurites and neurite length per cell. Particularly in the context of brain trauma, an increase in neurite outgrowth can have a positive effect in supporting brain functional connectivity and contribute to recovery of function. Any improvement, i.e., increase, in neurite outgrowth as observed in the PC12 cell study can result in an improvement in those parts of the brain that were affected by the trauma. The same principle applies to brain injury and/or brain damage.

Likewise, an increase in the density of neurite outgrowth in the context of neurodegenerative diseases can contribute to the compensation for lost neuronal connections in neurodegenerative diseases. It may allow surviving neurons to extend their neurites and establish new synaptic connections, thereby partially restoring functional circuits. This compensation can help mitigate the effects of neuronal loss and maintain or regain certain aspects of normal brain function, leading to improved prognosis and functional recovery. Furthermore, an increase in neurite outgrowth may contribute to neuroplasticity, allowing surviving neurons to adapt and establish new connections in response to the changing environment. This adaptability can help the brain compensate for the loss of specific neuronal populations and maintain functional connectivity, potentially slowing down disease progression and improving prognosis.

Without being bound by theory, it is believed that the administration of DHA together with vitamin D3 and vitamin B2 beneficially mitigates oxidative damage, thereby aiding in reducing the extent of neuronal damage and supporting brain metabolism, cell survival and providing anti-inflammatory action together supporting healing or recovery from neuronal damage. Choline is associated with decreased neuroinflammation, improved functional and structural connectivity, had beneficial effects on cerebral blood flow. Dietary fibres and/or dietary butyrate provide butyrate as alternative cellular energy source and exhibits neuroprotective effects by reducing oxidative stress and inflammation in neurons. The ingredients of the composition according to the invention are believed to provide on one hand an optimal promotion of neurite outgrowth and recovery after neuronal damage while at the same time supporting brain function.

The present invention thus concerns a method for treating neurodegenerative diseases and/or neurotrauma comprising administering to the subject a composition according to the invention. The invention further pertains to a composition comprising a combination of i) omega-3 fatty acid DHA and ii) vitamin B2 and iii) vitamin D3, and one or more of iv) choline and/or PC; and; v) dietary fibres and/or dietary butyrate for use in the treatment of neurodegenerative diseases and/or neurotrauma. In addition, the present invention concerns a method for treating brain injury and/or brain damage comprising administering to the subject the composition according to the invention. The invention further pertains to a composition comprising a combination of i) omega-3 fatty acid DHA and ii) vitamin B2 and iii) vitamin D3, and one or more of iv) choline and/or PC; and; v) dietary fibres and/or dietary butyrate for use in the treatment of brain injury and/or brain damage.

In some embodiments of the present invention, the dietary fibres are selected from short-chain galactooligosaccharides (scGOS), long-chain fructooligosaccharides (IcFOS) and low viscosity pectin. The invention thus pertains to a composition comprising a therapeutically effective combination of i) omega-3 fatty acid DHA ii) vitamin B2 iii) vitamin D3, further comprising iv) choline and/or PC and/or v) dietary fibres and/or dietary butyrate.

Preferably, the composition according to the invention further comprises one or more selected from A, B, C and E vitamin(s), phospholipids, selenium, magnesium, and co-enzyme Q10. In another preferred aspect the composition according to the invention comprises a combination of i) omega-3 fatty acid DHA, ii) vitamin B2, iii) vitamin D3, iv) choline and/or PC and v) dietary fibres and/or dietary butyrate and one or more further B vitamins.

In a further embodiment, the invention concerns a method for treating neurodegenerative diseases and/or neurotrauma in a subject, comprising administering to the subject the composition according to the invention. The invention may also be worded as the use of a therapeutically effective combination of i) omega-3 fatty acid DHA, ii) vitamin B2 and iii) vitamin D3 in combination with one or more of iv) choline and/or PC and v) dietary fibres and/or dietary butyrate for the manufacture of a composition for treating neurodegenerative diseases and/or neurotrauma. Also, the invention relates to the use of a therapeutically effective combination of i) omega-3 fatty acid DHA, ii) vitamin B2, and iii) vitamin D3, further comprising iv) choline and/or PC and/or v) dietary fibres and/or dietary butyrate for the manufacture of a composition for treating neurodegenerative diseases and/or neurotrauma in a subject. In other words, the invention concerns a composition for use in the treatment of neurodegenerative diseases and/or neurotrauma in a subject, said composition comprising a combination of i) omega-3 fatty acid DHA ii) vitamin B2 iii) vitamin D3, further comprising iv) choline and/or PC and/or v) dietary fibres and/or dietary butyrate.

In a further preferred embodiment, the invention concerns a method for treating brain injury and/or brain damage in a subject, comprising administering to the subject the composition according to the invention. The invention may also be worded as the use of the therapeutically effective combination of i) omega-3 fatty acid DHA, ii) vitamin B2 and iii) vitamin D3 in combination with one or more of iv) choline and/or PC and v) dietary fibres and/or dietary butyrate for the manufacture of a composition for treating brain injury and/or brain damage. Also, the invention relates to the use of a therapeutically effective combination of i) omega-3 fatty acid DHA, ii) vitamin B2, and iii) vitamin D3, further comprising iv) choline and/or PC and/or v) dietary fibres and/or dietary butyrate for the manufacture of a composition for treating brain injury and/or brain damage in a subject. In other words, the invention concerns a composition for use in the treatment of brain injury and/or brain damage in a subject, said composition comprising a combination of i) omega-3 fatty acid DHA ii) vitamin B2 iii) vitamin D3, further comprising iv) choline and/or PC and/or v) dietary fibres and/or dietary butyrate.

In an embodiment, the invention pertains to treating or preventing symptoms associated with neurodegenerative diseases, which involves, i.e. comprises, an improvement (e.g. increase in neurite number and neurite length) in neurite outgrowth in treated subjects that suffer from a neurodegenerative disease, as compared with subjects suffering from said neurodegenerative disease and not treated with the composition according to the invention. In a further embodiment, the invention pertains to treating neurotrauma which involves, i.e. comprises, an improvement (e.g. increase in neurite number and neurite length) in neurite outgrowth in treated subjects that suffer from neurotrauma, as compared with subjects not treated with the composition according to the invention. In a further embodiment, the invention pertains to treating brain injury and/or brain damage which comprises an improvement (e.g. increase in neurite number and neurite length) in neurite outgrowth in treated subjects that suffer from brain injury and/or brain damage as compared with subjects not treated with the composition according to the invention. In an alternative embodiment, the invention pertains to treating brain injury and/or brain damage which comprises improvement of neurological functioning selected from motor, behavioural, emotional, cognitive, sensory functioning in treated subjects that suffer from brain injury and/or brain damage as compared to subjects not treated with the composition according to the invention. In some embodiments, treatment of neurodegenerative diseases and/or neurotrauma further involves, i.e. comprises, neuroprotection after diagnosis with a neurodegenerative disease or neurotrauma. In a further embodiment, treatment of brain injury and/or brain damage comprises neuroprotection after diagnosis of said brain injury and/or brain damage.

The invention pertains to treating or preventing the symptoms associated with neurodegenerative diseases, which involves, i.e. comprises a prevention against or improvement in functional consequences of neurodegenerative diseases.

In some embodiments, the invention pertains to treating neurodegenerative diseases selected from Alzheimer's diseases, Parkinson's disease, Huntington's disease, frontotemporal dementia, and Lewy body dementia. In a further embodiment, the invention pertains to treating or preventing the symptoms associated with neurodegenerative diseases, which involves, i.e. comprises prevention against or improvement in functional consequences of neurodegenerative diseases, such as but not limited to
cognitive decline associated with memory loss, reduced attention span, reduced executive function, impairment in performing activities of daily living; and/or;
motor symptoms associated with tremors, rigidity, bradykinesia, coordination and balance;
behavioural and psychological changes associated with depression, anxiety, irritability, fatigue, apathy, social withdrawal and mood swings;
sensory disturbances associated with loss of sensation, numbness, tingling, or altered perception of touch, pain or temperature;
sleep disturbances associated with insomnia, excessive daytime sleepiness, fragmented sleep, restless legs syndrome and rapid eye movement (REM) sleep behaviour disorder;
autonomic dysfunction associated with orthostatic hypotension, urinary problems, incontinence, constipation, changes in sweating and body temperature regulation,

In a preferred embodiment, neurodegenerative diseases as defined herein are not caused by a stroke or an ischemic stroke.

In a further embodiment, the invention pertains to treating neurotrauma selected from traumatic brain injury (TBI), spinal cord injury (SCI), and peripheral nerve injuries. In some further embodiments, the invention pertains to treating neurotrauma caused by external factors selected from one or more of:
occupational hazards including car, sports, and workplace accidents;
explosions and blast injuries;
electric shock;
blunt force trauma;
penetrating injuries involving the penetration of an external object, including a bullet or a sharp object, into the brain, spinal cord, or peripheral nerves.

In a further embodiment, the invention pertains to treating brain injury and/or brain damage, wherein the treatment is selected from improving neurological recovery after brain injury and/or brain damage, preventing and/or reducing worsening of neurological impairment caused by brain injury and/or brain damage. Herein is meant as neurological recovery and neurological impairment, the neurological functioning of the brain selected from motor, behavioural, emotional, cognitive, sensory functioning. In a further embodiment, the invention pertains to treating brain injury and/or brain damage caused by internal factors selected from stroke, ischemia, bacterial and/or viral infection, diabetes mellitus type 1 or 2, acute alcohol poisoning, cancer treatment and chemotherapy. In a preferred embodiment, the brain injury and/or brain damage, is an acute injury meaning that the injury occurred within a time span of days, preferably within 21 days, more preferably within 15 days from the initiation of the treatment with the composition according to the invention. In a further preferred embodiment, the brain injury and/or brain damage is a local injury located at a specific or particular area of the brain. In a more preferred embodiment, the invention pertains to treating brain injury and/or brain damage, wherein the brain injury and/or brain damage is a local and acute injury in the brain.

In an alternative embodiment, the invention pertains to treating cerebral palsy.

In a preferred embodiment, the administration or use of the composition is initiated after a neurodegenerative disease or the prodromal phase thereof has been diagnosed and/or after neurotrauma has occurred and the administration or use of the composition continues for a period of at least 2 weeks, preferably at least 1 month, preferably at least 2 months, more preferably at least 4 months, even more preferably at least 6 months. In a further preferred embodiment, the administration or use of the composition is initiated after brain injury and/or brain damage has occurred and the administration of use of the composition continues for a period of at least 2 weeks, preferably at least 1 month, preferably at least 2 months, more preferably at least 4 months, even more preferably at least 6 months.

### LIST OF FIGURES

The present invention will be discussed in more detail below, with reference to the attached figures. In all figures neurite outgrowth is expressed as percentage relative to control (100%).

FIG. 1. The effects of omega-3 fatty acids DHA (6.7 μM) and eicosapentaenoic acid (EPA) (10 μM), vitamin B2 (1.25 μM) and vitamin D3 (0.25 μM) and their combination on neurite outgrowth in PC12 neuronal cells are shown.

FIG. 2. The effects of the combination of omega-3 fatty acid DHA (6.7 μM), vitamin B2 (0.5 μM) and vitamin D3 (0.25 μM) and the combination of DHA (6.7 μM), vitamin B2 (1 μM) and vitamin D3 (0.25 μM on neurite outgrowth in PC12 neuronal cells are shown.

FIG. 3. The effects of the combination of omega-3 fatty acid DHA (3.3 μM), vitamin B2 (0.5 μM) and vitamin D3 (0.25 μM) and the combination of DHA (3.3 μM), vitamin B2 (1 μM) and vitamin D3 (0.25 μM) on neurite outgrowth in PC12 neuronal cells are shown.

FIG. 4. The effects of choline-chloride (20 μM), PC (20μ) and their combination providing for 20 μM choline in a 60:40 respectively 40:60 molar ratio on neurite outgrowth in PC12 neuronal cells are shown.

FIG. 5. The effects of choline-chloride (20 μM), PC (20 μM) alone and their combination with DHA (3.3 μM), vitamin B2 (0.5 μM) and vitamin D3 (0.25 μM) on neurite outgrowth in PC12 neuronal cells are shown. The effect of a combination of choline-chloride and phosphatidylcholine providing for 20 μM choline wherein 80 mol % of choline is derived from choline-chloride and 20 mol % is derived from PC based on total choline, with DHA, vitamin B2 and vitamin D3 is also shown.

FIG. 6. The effects of PC (20 μM), the combination of DHA (5 μM) and EPA (1 μM), vitamin B2 (0.5 μM) and vitamin D3 (0.25 μM) and the combination of PC together with DHA, EPA, vitamin B2 and vitamin D3 on neurite outgrowth in PC12 neuronal cells are shown.

FIG. 7. The effects of butyric acid (BA) in different concentrations on neurite outgrowth in PC12 neuronal cells are shown.

FIG. 8. The effects of BA (200 μM), vitamin B2 (0.25 μM), DHA (2.5 μM), vitamin D3 (0.25 μM) and PC (20 μM) alone on neurite outgrowth in PC12 neuronal cells are shown. The effects of a combination of DHA, vitamin B2 and D3, a combination of DHA, vitamin B2 and D3 and with either BA or PC and the combination of all nutrients BA, DHA, PC, vitamin B2 and D3 are also shown.

FIG. 9. The effects of vitamin B2 (0.25 μM), vitamin D3 (0.75 μM), PC, (15 μM), and BA (40 μM) alone on neurite outgrowth in PC12 neuronal cells are shown. The effect of a reference diet comprising PC (5 μM), DHA (2 μM), choline-chloride (4 μM), vitamin C (15 μM), vitamin E (4 μM), vitamin B6 (2 μM), vitamin B9 (3 μM), vitamin B12 (0.02 μM) and selenium (0.016 μM) is also shown. The combination of the reference diet with further supplementation of vitamin B2 (0.25 μM), vitamin D3 (0.75 μM), PC, (15 μM), and BA (40 μM) is further shown.

FIG. 10. The effects of vitamin B2 (0.25 μM), vitamin D3 (0.75 μM) and BA (200 μM) alone on neurite outgrowth in PC12 neuronal cells are shown. The effect of a reference diet comprising PC (5 μM), DHA (2 μM), choline-chloride (4 μM), vitamin C (15 μM), vitamin E (4 μM), vitamin B6 (2 μM), vitamin B9 (3 μM), vitamin B12 (0.02 μM) and selenium (0.016 μM) is also shown. The combination of the reference diet with further supplementation of vitamin B2 (0.25 μM), vitamin D3 (0.75 μM) and BA (200 μM) is further shown.

FIG. 11. The mNSS (Modified Neurological Severity Score) scores are shown at baseline (BL) and over a timespan of 7 weeks post-surgery with three study groups consisting of a group with sham injury and given the Control diet, a group with brain injury and given the Control diet and a group with brain injury given the Active diet.

FIG. 12. The foot-slip test results at 51 days post-surgery are shown with three study groups consisting of a group with sham injury and given the Control diet, a group with brain injury and given the Control diet and a group with brain injury given the Active diet.

### LIST OF PREFERRED EMBODIMENTS

1. A nutritional composition comprising a therapeutically effective combination of
i) omega-3 fatty acids, wherein the omega-3 fatty acids comprises at least DHA;
ii) vitamin B2; and
iii) vitamin D3,
   and one or more selected from
iv) choline or a salt thereof and/or phosphatidylcholine; and
v) dietary fibres and/or dietary butyrate,
   for use in the treatment of neurodegenerative diseases and/or neurotrauma and/or cerebral palsy, preferably for use in the treatment of neurodegenerative diseases and/or neurotrauma, brain injury and/or brain damage and/or cerebral palsy.

2. The nutritional composition for use according to embodiment 1, wherein at least 40 mol % choline is provided in the form of phosphatidylcholine, preferably the molar ratio of choline or a salt thereof to PC is in the range from 40:60 to 60:40.

3. The nutritional composition for use according to any one of embodiments 1-2, further comprising one or more components selected from vitamin A, C and E, phospholipids, selenium, magnesium, and further B vitamins.

4. The nutritional composition for use according to any one of the preceding embodiments, wherein the neurodegenerative disease is selected from Alzheimer's diseases, Parkinson's disease, Huntington's disease, frontotemporal dementia and Lewy body dementia.

5. The nutritional composition for use according to any one of the preceding embodiments, wherein the treatment of a neurodegenerative disease involves, i.e. comprises treating and/or preventing and/or delaying the symptoms associated with said neurodegenerative disease in a subject that is suffering from said neurodegenerative disease or wherein the prodromal phase of said neurodegenerative disease has been diagnosed in said subject, preferably wherein the symptoms involve, i.e comprises, one or more of:
cognitive decline associated with memory loss, reduced attention span, reduced executive function, impairment in performing activities of daily living; and/or
motor symptoms associated with tremors, rigidity, bradykinesia, coordination, and balance; and/or
behavioural and psychological changes associated with depression, anxiety, irritability, fatigue, apathy, social withdrawal, and mood swings; and/or
sensory disturbances associated with loss of sensation, numbness, tingling, or altered perception of touch, pain or temperature; and/or
sleep disturbances associated with insomnia, excessive daytime sleepiness, fragmented sleep, restless legs syndrome and rapid eye movement (REM) sleep behaviour disorder; and/or
autonomic dysfunction associated with orthostatic hypotension, urinary problems, incontinence, constipation, changes in sweating and body temperature regulation,

6. The nutritional composition for use according to embodiment 1, wherein the neurotrauma is selected from traumatic brain injury (TBI), spinal cord injury (SCI), and peripheral nerve injuries.

7. The nutritional composition for use according to embodiment 6, wherein the neurotrauma is caused by external factors selected from one or more of:
occupational hazards including car, sports, and workplace accidents;
explosions and blast injuries;
electric shock;
blunt force trauma;
penetrating injuries involving the penetration of an external object, including a bullet or a sharp object, into the brain, spinal cord or peripheral nerves.

8. The nutritional composition for use according to any one of embodiments 1 to 7, wherein the treatment of neurodegenerative diseases involves, i.e. comprises an increase in neurite outgrowth in subjects suffering from a neurodegenerative disease and treated with the composition as compared to subjects suffering from said neurodegenerative disease and not treated with the composition and/or wherein the treatment of neurotrauma involves, i.e. comprises an increase in length of neuronal outgrowth in subjects suffering from neurotrauma and treated with the composition as compared to subjects suffering from said neurotrauma and not treated with the composition, preferably wherein the treatment of neurotrauma, brain injury and/or brain damage comprises an increase in length of neuronal outgrowth in subjects suffering from neurotrauma, brain injury and/or brain damage and treated with the composition as compared to subjects suffering from said neurotrauma, brain injury and/or brain damage and not treated with the composition.

9. The nutritional composition for use according to any one of the preceding embodiments, wherein the composition is used daily for at least 4 weeks after diagnosis of said neurodegenerative disease and/or after diagnosis or occurrence of said neurotrauma, preferably after diagnosis of said neurodegenerative disease and/or diagnosis or occurrence of said neurotrauma, brain injury and/or brain damage.

10. The nutritional composition for use according to any one of the preceding embodiments, wherein the composition comprises daily dosages of:
i) 150 to 1200 mg, preferably 200 to 1100 mg, more preferably 250 to 1000 mg DHA per day;
ii) 0.8 to 7.6 mg, preferably 1 to 7.4 mg, more preferably in the range of 1.2 to 7.2 mg vitamin B2;
iii) 6 to 75 μg, preferably 8 to 70 μg, more preferably 10 to 65 μg vitamin D3;
and one or more of:
iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and/or
v) 2.5 to 12.5 g, preferably 3 to 12 g, more preferably 3.5 to 11.5 g dietary fibres and/or 0.125 g to 0.65 g, preferably 0.025 g to 0.65 g, more preferably 0.043 g to 0.65 g dietary butyrate.

11. The nutritional composition for use according to any one of the preceding embodiments wherein the nutritional composition is a liquid nutritional composition, preferably wherein the nutritional composition is an oral nutritional supplement or a tube feed.

12. A liquid nutritional composition comprising a combination of
i) DHA;
ii) vitamin B2;
ii) vitamin D3;
   and one or more from
iv) choline or a salt thereof and/or phosphatidylcholine; and
v) dietary fibres and/or dietary butyrate.

13. Liquid nutritional composition according to embodiment 12, wherein the composition comprises per 100 kcal
i) 20 to 100 mg, preferably 25 to 90 mg, more preferably 30 to 80 mg DHA;
ii) 0.4 to 0.6 mg vitamin B2;
iii) 1.3 to 4.5 μg vitamin D3;
and further comprises
iv) 100 to 1000 mg, preferably 200 to 800 mg, more preferably 300 to 500 mg choline or a salt thereof and/or phosphatidylcholine;
and/or
v) 0.75 to 2.5 g, preferably 1.0 to 2.0 g, more preferably 1.1 to 1.5 g dietary fibres and/or 0.015 to 0.25 g, preferably 0.03 to 0.25 g, more preferably 0.05 to 0.25 g dietary butyrate.

14. Liquid nutritional composition according to embodiments 12 or 13, wherein the composition additionally comprises 4 to 7 g protein per 100 kcal, 3 to 5.5 g lipids per 100 kcal and 8.5 to 10.5 g carbohydrates per 100 kcal.

### DETAILED DESCRIPTION

The invention pertains to a composition comprising a combination of i) omega-3 fatty acid DHA, ii) vitamin B2, iii) vitamin D3, and further comprising iv) choline and/or PC and/or v) dietary fibres and/or dietary butyrate. Preferably, the composition further comprises one or more selected from A, B, C and E vitamin(s), phospholipids, selenium, magnesium, and co-enzyme Q10. In another preferred aspect the composition according to the invention comprises a combination of i) omega-3 fatty acid DHA ii) vitamin B2 iii) vitamin D3 iv) choline and/or PC and v) dietary fibres and/or dietary butyrate, and one or more further B vitamins.

The present invention further concerns a method for treating neurodegenerative diseases and/or neurotrauma comprising administering to a subject a composition comprising a combination of i) omega-3 fatty acid DHA ii) vitamin B2 and iii) vitamin D3, further comprising one or more of iv) choline and/or PC and v) dietary fibres and/or dietary butyrate. The invention further concerns a method for treating brain injury and/or brain damage comprising administering to a subject a composition comprising a combination of i) omega-3 fatty acid DHA ii) vitamin B2 and iii) vitamin D3, further comprising one or more of iv) choline and/or PC and v) dietary fibres and/or dietary butyrate.

In a preferred embodiment dietary butyrate is selected from butyrate and its salts and glyceride bound forms of butyrate comprising one, two or three butyrate chains. In a preferred embodiment dietary fibres are selected from pectins, mucilages, gums, GOS, oligofructan, inulin, polyfructoses, FOS, arabinogalactans, (hemi) cellulose, resistant starch, soy fiber, oligosaccharides, and mixtures thereof. Preferably the dietary fibers comprise or consist of scGOS, IcFOS, and low viscosity pectin.

Alternatively worded, the invention also concerns the use of a composition comprising a combination of i) omega-3 fatty acid DHA ii) vitamin B2 iii) vitamin D3, further comprising iv) choline and/or PC and/or v) dietary fibres and/or dietary butyrate for the manufacture of a composition for treating neurodegenerative diseases and/or neurotrauma in a subject, preferably for treating neurodegenerative diseases selected from Alzheimer's diseases, Parkinson's disease, Huntington's disease, frontotemporal dementia and Lewy body dementia and for treating neurotrauma selected from traumatic brain injury (TBI), spinal cord injury (SCI), and peripheral nerve injuries. The invention also concerns the use of a composition comprising a combination of i) omega-3 fatty acid DHA ii) vitamin B2 iii) vitamin D3, further comprising iv) choline and/or PC and/or v) dietary fibres and/or dietary butyrate for the manufacture of a composition for treating brain injury and/or brain damage selected from improving neurological recovery after brain injury and/or brain damage, preventing and/or reducing worsening of neurological impairment caused by brain injury and/or brain damage. Preferably, the composition according to the invention further comprises one or more selected from A, B, C and E vitamin(s), phospholipids, selenium, magnesium, and co-enzyme Q10.

Also, the invention pertains to a composition for use in the treatment of neurodegenerative diseases and/or neurotrauma, wherein the neurodegenerative diseases are preferably selected from diseases selected from Alzheimer's diseases, Parkinson's disease, Huntington's disease, frontotemporal dementia and Lewy body dementia and wherein neurotrauma is selected from traumatic brain injury (TBI), spinal cord injury (SCI), and peripheral nerve injuries, said composition comprising a combination of i) omega-3 fatty acid DHA, ii) vitamin B2, iii) vitamin D3 and further comprising iv) choline and/or PC and/or v) dietary fibres and/or dietary butyrate. Also, the invention pertains to a composition for use in the treatment of brain injury and/or brain damage, wherein the treatment of brain injury and/or brain damage is selected from improving neurological recovery after brain injury and/or brain damage, preventing and/or reducing worsening of neurological impairment caused by brain injury and/or brain damage, said composition comprising a combination of i) omega-3 fatty acid DHA, ii) vitamin B2, iii) vitamin D3 and further comprising iv) choline and/or PC and/or v) dietary fibres and/or dietary butyrate. Preferably, the composition according to the invention further comprises one or more selected from A, B, C and E vitamin(s), phospholipids, selenium, magnesium, and co-enzyme Q10. In a particular preferred embodiment, the composition comprises a combination of i) omega-3 fatty acid DHA, ii) vitamin B2, iii) vitamin D3, iv) choline and/or PC and v) dietary fibres and/or dietary butyrate, and one or more further B vitamins.

The invention further pertains to treating neurodegenerative diseases, more in particular treating, reducing, delaying or preventing the symptoms of neurodegenerative diseases, wherein the neurodegenerative disease is preferably selected from Alzheimer's diseases, Parkinson's disease, Huntington's disease, frontotemporal dementia and Lewy body dementia, In a preferred embodiment, treatment of neurodegenerative diseases involves or comprises treating, reducing, delaying and/or preventing the symptoms associated with said neurodegenerative diseases in a subject that is suffering from or in the prodromal phase for neurodegenerative diseases, wherein the symptoms involve one or more of:
cognitive decline associated with memory loss, reduced attention span, reduced executive function in performing activities of daily living;
motor symptoms associated with tremors, rigidity, bradykinesia, coordination, and balance;
behavioural and psychological changes associated with depression, anxiety, irritability, fatigue, apathy, social withdrawal, and mood swings;
sensory disturbances associated with loss of sensation, numbness, tingling, or altered perception of touch, pain or temperature;
sleep disturbances associated with insomnia, excessive daytime sleepiness, fragmented sleep, restless legs syndrome and rapid eye movement (REM) sleep behaviour disorder;
autonomic dysfunction associated with orthostatic hypotension, urinary problems, incontinence, constipation, changes in sweating and body temperature regulation.

The treatment of neurodegenerative diseases, preferably Alzheimer's diseases, Parkinson's disease, Huntington's disease, frontotemporal dementia, and Lewy body dementia, involves, i.e. comprises an improvement in neurite outgrowth and wherein the improvement in neurite outgrowth comprises an increase in the number of neurites and neurite length per cell in subjects treated with the composition according to the invention, as compared with subjects not treated with the composition according to the invention.

In some embodiments, treatment of neurodegenerative diseases, preferably Alzheimer's diseases, Parkinson's disease, Huntington's disease, frontotemporal dementia, and Lewy body dementia, further involves, i.e. comprises neuroprotection after diagnosis. The treatment of neurodegenerative diseases thus involves, i.e. comprises neuronal repair or regeneration and recovery of neuronal networks in neurodegenerative diseases. In an embodiment, the treatment of neurodegenerative diseases includes neuronal repair and/or recovery of neuronal networks. In an embodiment the treatment involves, i.e. comprises treatment of the symptoms and/or delaying the progression of a neurodegenerative diseases. Furthermore, the invention further pertains to treating neurotrauma, preferably traumatic brain injury (TBI), spinal cord injury (SCI), and peripheral nerve injuries. In some further embodiments, the invention pertains to treating neurotrauma caused by external factors selected from one or more of:
occupational hazards including car, sports, and workplace accidents;
explosions and blast injuries;
electric shock;
blunt force trauma;
penetrating injuries involving the penetration of an external object, including a bullet or a sharp object, into the brain, spinal cord or peripheral nerves.

The treatment of neurotrauma, preferably traumatic brain injury (TBI), spinal cord injury (SCI), and peripheral nerve injuries, involves, i.e. comprises an improvement in neurite outgrowth referring to an increase in the number of neurites and neurite length per cell in subjects treated with the composition according to the invention as compared with subjects not treated with the composition according to the invention. In some embodiments, treatment of neurotrauma, preferably traumatic brain injury (TBI), spinal cord injury (SCI), and peripheral nerve injuries, further involves, i.e. comprises neuroprotection after diagnosis. The treatment of neurotrauma thus involves, i.e. comprises neuronal repair or regeneration and recovery of neuronal networks after neurotrauma has occurred. In an embodiment, the treatment of neurotrauma includes neuronal repair and/or recovery of neuronal networks. In an embodiment the treatment of neurotrauma comprises treatment of the consequences of and/or prevention of progression of neuronal damage as a consequence of neurotrauma.

Furthermore, the invention further pertains to treating brain injury and/or brain damage, preferably selected from improving neurological recovery after brain injury and/or brain damage, preventing and/or reducing worsening of neurological impairment caused by brain injury and/or brain damage. Neurological recovery and neurological impairment as used herein comprise improving, the neurological functioning of the brain selected from motor, behavioural, emotional, cognitive and sensory functioning. In some further embodiments, the invention particularly pertains to treating brain injury and/or brain damage caused by internal factors selected from stroke, ischemia, bacterial and/or viral infection, diabetes mellitus type 1 or 2, acute alcohol poisoning, cancer treatment and chemotherapy. In a preferred embodiment, the brain injury and/or brain damage, is an acute injury meaning that the injury occurred within a time span of days, preferably within 21 days, more preferably 15 days. In a further preferred embodiment, the brain injury and/or brain damage is a local injury located at a specific or particular area of the brain. In a more preferred embodiment, the invention pertains to treating brain injury and/or brain damage, wherein the brain injury and/or brain damage is a local and acute injury in the brain. The method or use or composition for use according to the invention is further described in the following embodiments.

### Definitions

Throughout this application, the following terminology and abbreviations may be used. “Nutritional composition” means a substance or formulation that satisfies at least a portion of a subject's nutrient requirements. The terms “nutritional(s)”, “nutritional formula(s)”, “enteral nutritional(s)”, and “nutritional supplement(s)” are used as non-limiting examples of nutritional composition(s) throughout the present disclosure. Moreover, “nutritional composition(s)” may refer to liquids, powders, gels, pastes, solids, concentrates, suspensions, or ready-to-use forms of enteral formulas, oral formulas, formulas for infants, formulas for pediatric subjects, formulas for children, growing-up milks and/or formulas for adults.

The term “supplement” or “nutritional supplement” refers to a nutritional product that provides nutrients to an individual that may otherwise not conveniently be consumed in sufficient quantities by said individual and may be used to complement the nutrition of an individual. It may be in the form of tablets, capsules, pastilles or a liquid and the like. Supplements typically provide the selected nutrients while not representing a significant portion of the overall nutritional needs of the subject, not representing more than 0.1%, 1%, 5%, 10% of the daily energy need of the subject.

As used herein, “neuroprotection” refers to promotion of neuronal repair, limiting neuronal oxidative stress, limiting cell damage and/or promotion of recovery of neuronal networks after neurotrauma or during neurodegenerative diseases.

“Prevention” includes reduction of risk and/or severity of neuronal damage from neurodegenerative diseases. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition in a subject in need thereof.

The subject as used herein is a mammalian subject, preferably a human, more preferably a human subject older than 40 years, preferably older than 55 years, more preferably older than 65 years.

As used herein, a “therapeutically effective amount” is an amount that reduces symptoms, manages progression of the consequences of neurodegenerative diseases and/or neurotrauma, brain injury, and/or brain damage or provides a nutritional, physiological, or medical benefit to the individual. In a preferred embodiment, therapeutically effective amounts as used herein refers to amounts of at least 1, preferably 1.1, even more preferably 1.5, most preferably 1.7 times the amount of the recommended daily intake. The recommended daily intakes are defined by authorities such as EFSA or FDA.

The term “neurite” refers to processes growing out of a neuron. The term neurite as used herein encompasses all such cell processes (including both axons and dendrites) growing out of a neuron.

The term “neurite outgrowth” refers to the process of cells growing out of a neuron, or to the cells comprising an outgrowth from a neuron and herein refers to the number of neurites and neurite length per cell.

The term “butyric acid” and “butyrate”, also referred to as BA are used interchangeably and refers to a short-chain fatty acid (SCFA) with a four-carbon chain, that can be covalently bound to another compound with e.g., an ester bond, or the anion thereof.

In this document and in its claims, the verb “to comprise” and its conjugations is used in its non-limiting sense to mean that items following the word are included, but items not specifically mentioned are not excluded. Further when used herein the term weight percent (wt %) is the percentage of weight based on the total weight. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

### Composition

The method or use or composition for use according to the invention involves administration of the composition according to the invention. The composition according to the invention may be used as a pharmaceutical product or preferably a nutritional product. In one embodiment the present nutritional composition is a liquid. In one embodiment, preferably the present nutritional composition is a liquid ready-to-feed composition. Preferably the composition is administered orally or as tube feed.

In one aspect, the composition according to the invention may be used as a pharmaceutical product comprising one or more pharmaceutically acceptable carrier materials. Such product may contain the daily dosages as defined below in one or more dosage units. The dosage unit may be in a liquid form or in a solid form, wherein in the latter case the daily dosage may be provided by one or more solid dosage units, e.g. in one or more capsules or tablets. The pharmaceutical product, preferably for enteral application, may be a solid or liquid galenical formulation. Examples of solid galenical formulations are tablets, capsules (e.g. hard- or soft-shell gelatine capsules), pills, sachets, powders, granules and the like which contain the active ingredients together with conventional galenical carriers. Any conventional carrier material can be utilized. The carrier material can be organic or inorganic inert carrier material suitable for oral administration. Suitable carriers include water, gelatine, gum Arabic, lactose, starch, magnesium stearate, talc, vegetable oils, and the like. Additionally, additives such as thickeners, flavouring agents, preservatives, stabilizers, emulsifying agents, buffers, and the like may be added in accordance with accepted practices of pharmaceutical compounding. While the individual active ingredients are suitably administered in a single composition, they may also be administered in individual dosage units.

In a preferred aspect, the composition according to the invention may be used as a nutritional product, for example as a nutritional supplement, e.g. as an additive to a normal diet, as a fortifier, to add to a normal diet, or as a complete nutrition. The nutritional product preferably comprises at least one component, preferably all components, selected from the group of fats, proteins, and carbohydrates. It is understood that a nutritional product differs from a pharmaceutical product by the presence of nutrients which provide nutrition to the subject to which the composition is administered, in particular the presence of protein, fat, digestible carbohydrates and dietary fibres. It may further contain ingredients such as minerals, vitamins, organic acids, and flavouring agents. Although the term “nutraceutical product” is often used in literature, it denotes a nutritional product with a pharmaceutical component or pharmaceutical purpose. Hence, the nutritional composition according to the invention may also be used in a nutraceutical product.

In one embodiment, the composition comprises a lipid fraction, wherein the lipid fraction provides between 15 and 50 energy % of the composition. In one embodiment, the composition is a liquid composition containing between 1.1 and 2.6 kcal per ml, preferably between 1.2 and 2.4 kcal per ml. In one embodiment the composition is a tube feed having a caloric density of between 1.1 and 1.7 kcal per ml, preferably between 1.2 and 1.6 kcal per ml. In another embodiment the composition is in the form of an oral nutritional supplement having a caloric density of between 2.2 and 2.6 kcal per ml. The energy provided by nutrients is calculated using the Atwater calculation factors of 9 kcal per g lipid, 4 kcal per gram protein or gram digestible carbohydrate, 2 kcal per gram fiber and zero kcal for the other components in the product.

The amount of lipid fraction can be determined by applying the methods known in the art for measuring fat content in the food matrix as applicable. For example, fat content for general foods is determined by applying AOAC® official method 983.23, while the Roese-Gottlieb method (AOAC® 932.06) is better applicable for products based on dried milk (Lehner, R., Estoppey, A., (1954) Mitt. Lebensmitteluntersuchung Hyg. 54:183-185). The amount of individual lipid components can be determined by applying methods specifically designed for measuring that specific component or by fractionating the fat fraction isolated from the extraction of the chloroform-niethanol fraction as given in the 983.23 method.

Suitable sources of omega-3 fatty acids for use in the composition include but are not limited to fish oil, krill oil, algae oil, preferably fish oil. Suitable sources of further lipids for use in the composition include but are not limited to soybean oil, rape seed oil (such as colza oil, low erucic acid rape seed oil and canola oil), high oleic sunflower oil, coconut oil, high oleic safflower oil and olive oil. An alternative option are lipids obtained from milk from non-human mammals, preferably cow's milk, goat milk, sheep milk, horse milk, buffalo milk, yak milk, reindeer milk, donkey milk and camel milk, particularly cow's milk and/or goat milk. Milk lipid is sometimes also referred to as milk fat or butter fat.

The nutritional composition according to the invention thus preferably comprises a lipid fraction, preferably a lipid fraction suitable for nutrition as known in the art. The composition comprises 4 to 12 g lipid per 100 ml, preferably 4.5 to 11 g lipid per 100 ml, more preferably 5 to 10 g per 100 ml. Based on calories, preferably the composition according to the invention may comprise 3 to 5.5 g of lipid per 100 kcal, preferably 3.5 to 5 g per 100 kcal, more preferably 3.8 to 4.5 g of lipid per 100 kcal based on the total energy content of the composition. The lipid fraction includes polyunsaturated fatty acids and optionally also mono-unsaturated fatty acids (MUFAS) and saturated fatty acids (SFA).

The composition according to the invention preferably comprises 4 to 17 g of protein per 100 ml of the composition. The total protein that is present in the nutritional composition, i.e., the combination of all proteins present, may also be referred to as the “protein fraction” of the nutritional composition. The nutritional composition thus preferably comprises a protein fraction of 4-17 g per 100 ml of the composition. In a preferred aspect the nutritional composition comprises a protein fraction of 5 to 16 g per 100 ml of the composition, more preferably of 6-15 g. Most preferably the composition comprises a protein fraction of 11 to 14.5 g per 100 ml of the composition. Based on calories, preferably the composition according to the invention may comprise 4 to 7 g of protein fraction per 100 kcal, preferably 4.5 to 6.5 g of protein fraction per 100 kcal, more preferably 5 to 6 g of protein fraction per 100 kcal based on the total energy content of the composition.

Suitable protein sources may be based on cows' milk proteins such as whey, casein, and mixtures thereof and vegetable proteins such as those of soy, potato, pea, rapeseed and the like. The nutritional composition preferably further comprises a carbohydrate fraction. The composition preferably comprises 9 to 25 g carbohydrates per 100 ml, more preferably 10 to 24 g per 100 ml, even more preferably 11 to 23 per 100 ml. Based on calories, preferably the composition according to the invention may comprise 8.5 to 10.5 g of carbohydrates per 100 kcal, preferably 9 to 10 g per 100 kcal, more preferably 9.2 to 9.6 g of carbohydrates per 100 kcal based on the total energy content of the composition. The composition according to the invention preferably comprises 4 to 7 g protein per 100 kcal, 3 to 5.5 g lipids per 100 kcal and 8.5 to 10.5 g carbohydrates per 100 kcal.

Suitable carbohydrate sources include lactose, glucose, sucrose, fructose, galactose, maltose, starch, isomaltulose, and maltodextrin.

The composition of the invention is typically an enteral composition, i.e., intended for oral administration such as a tube feed, a nutritional drink, and an oral nutritional supplement. It is preferably administered in liquid form. Preferably, the composition comprises water in which the further components are dissolved or suspended. The composition is thus preferably a liquid, or a solid (typically a powder or tablet, preferably a powder) which is reconstitutable with a liquid, preferably water, to obtain a liquid composition. Dosages of components defined below may for example be in daily dose or in a concentration per 100 mL or 100 kcal.

The composition according to the invention is for treating neurodegenerative diseases and/or neurotrauma. Also, the composition according to the invention is for treating brain injury and/or brain damage. The neurodegenerative disease is preferably selected from Alzheimer's diseases, Parkinson's disease, Huntington's disease, frontotemporal dementia, and Lewy body dementia. Neurotrauma is preferably selected from traumatic brain injury (TBI), spinal cord injury (SCI), and peripheral nerve injuries. The treatment involves, i.e. comprises treatment of the symptoms and/or delaying the progression of a neurodegenerative disease and treatment of the consequences of and/or preventing progression of neuronal damage as a consequence of neurotrauma. Neurotrauma includes trauma caused by external factors such as occupational hazards; explosions and blast injuries; electric shock; blunt force trauma and penetration of external objects into the brain, spinal cord or peripheral nerves. The treatment involves, i.e. comprises an improvement in neurite outgrowth. The brain injury and/or brain damage is preferably improving neurological recovery after brain injury and/or brain damage, preventing and/or reducing worsening of neurological impairment caused by brain injury and/or brain damage. The treatment particularly comprises treatment of the consequences of and/or preventing progression of neuronal damage as a consequence of brain injury and/or brain damage. Brain injury and/or brain damage includes injury or damage caused by internal factors selected from stroke, ischemia, bacterial and/or viral infection, diabetes mellitus type 1 or 2, acute alcohol poisoning, cancer treatment and chemotherapy. The treatment comprises an improvement in neurite outgrowth and/or improvement in neurological functioning selected from one or more of motor, cognitive, behavioral, emotional and sensory functioning.

The compositions as described above can be used as a nutritional therapy, nutritional support, as a medical food, as a food for special medical purposes or as a nutritional supplement. Administration of the composition according to the invention typically occurs after diagnosis of a neurodegenerative disease and/or during recovery and/or rehabilitation after the occurrence of neurotrauma, and may be continued as long as negative effects thereof prolong. Also, administration of the composition according to the invention typically occurs during recovery and/or rehabilitation after the occurrence of brain injury and/or brain damage and may be continued as long as negative effects thereof prolong. Although positive effects can already be observed during the first week of administration, administration is preferably continued for at least 2 weeks, preferably at least 4 weeks, preferably at least 2 months, more preferably at least 4 months, even more preferably at least 6 months. Administration preferably starts at the first day after diagnosis of a neurodegenerative disease and/or after occurrence/diagnosis of neurotrauma. Also, administration preferably starts at the first day after occurrence/diagnosis of brain injury and/or brain damage.

The composition according to the Invention can be consumed in the form of a liquid composition, preferably an oral nutritional supplement at one, two or three servings of 50-250 ml per day, typically servings of 125 mL each, after diagnosis with a neurodegenerative disease and/or during recovery and/or rehabilitation after the occurrence of a neurotrauma. Also, the composition according to the invention can be consumed in the form of a liquid composition, preferably an oral nutritional supplement at one, two or three servings of 50-250 mL per day, typically servings of 125 mL each, during recovery and/or rehabilitation after the occurrence of brain injury and/or brain damage. Preferred daily dosages are in the range of 100 to 500 mL, more preferably 125 to 375 mL, most preferably 200 to 300 mL. Preferably, the composition is enterally administered. Administration occurs preferably at least one time per day, although alternative dosage regimes can be determined from these numbers. Alternatively, the composition may be a tube feed and is administered continuously. Preferred daily dosages of a tube feed are in the range of 1000 to 3000 kcal per day, preferably 1500 kcal to 2500 kcal per day. Preferably about 1 to 2 liters of tube feed is administered per day.

The method or use or composition for use according to the invention relates to composition comprising a combination of i) omega-3 fatty acid DHA and ii) vitamin B2 and iii) vitamin D3 and further comprises one or more of iv) choline and/or PC; and; v) dietary fibres and/or dietary butyrate. In an embodiment, the composition further comprises one or more selected from A, B, C and E vitamin(s), phospholipids, selenium, magnesium and co-enzyme Q10. In another preferred aspect the composition according to the invention comprises a combination of i) omega-3 fatty acid DHA ii) vitamin B2 iii) vitamin D3 iv) choline and/or PC and v) dietary fibres and/or dietary butyrate and one or more further B vitamins.

### Omega-3 Fatty Acids

The present composition according to the invention comprises omega-3 polyunsaturated fatty acids (PUFA), wherein the PUFAs are n-3 LC-PUFAs (long-chain PUFAs). In an embodiment, the composition comprises at least docosahexaenoic acid (22:6, ω-3; DHA). Preferably the composition comprises two omega-3 long-chain polyunsaturated fatty acids (LC-PUFA; having a chain length of 18 and more carbon atoms) wherein one omega-3 fatty acid is DHA and the second omega-3 fatty acid is selected from eicosapentaenoic acid (20:5, ω-3; EPA) and docosapentaenoic acid (22:5 ω-3; DPA), preferably the second omega-3 fatty acid is EPA. EPA is converted to DPA (ω-3), increasing subsequent conversion of DPA to DHA in the brain. In an alternative embodiment, the composition comprises only DHA as omega-3 fatty acid and preferably comprises no EPA.

In an embodiment, the composition comprises omega-3 fatty acid DHA, vitamin B2 and vitamin D3, optionally further comprising EPA as omega-3 fatty acid. In a further embodiment, the composition according to the invention may comprise a combination of DHA, vitamin B2, vitamin D3, in combination with EPA and further comprising one or more of: choline and/or PC; and dietary fibres and/or dietary butyrate. In a further embodiment the composition according to the invention may comprise a combination of omega-3 fatty acids DHA and EPA, vitamin B2, vitamin D3, choline and one or more of dietary fibres and dietary butyrate.

The DHA, EPA and/or DPA may be provided in any form such as, but not limited to, triglycerides, diglycerides, monoglycerides, free fatty acids or their salts or esters, phospholipids, lysophospholipids, glycerol ethers, lipoproteins, ceramides, glycolipids or combinations thereof. Preferably, the present composition comprises at least DHA in triglyceride form. Suitable omega-3 LCPUFA and/or sources of DHA and EPA include tuna oil, (other) fish oils, DHA-rich alkyl esters, algae oil, egg yolk, krill oil or phospholipids enriched with omega-3 LCPUFA e.g. phosphatidylserine-DHA. Preferably, the composition or composition for use according to the invention comprises fish oil providing the omega-3 LCPUFA(s).

DHA is preferably administered in an amount of 100 to 1250 mg per day, more preferably 150 to 1200 mg per day, even more preferably 200 to 1100 mg per day, most preferably 250 to 1000 mg per day. In an embodiment when the composition is in the form of a tube feed the daily amount of DHA administered is preferably 200 to 1100 mg per day, more preferably 250 to 1050 mg per day, most preferably 300 to 1000 mg per day. The present composition preferably comprises in the range of 480-680 mg DHA, more preferably 510-650 mg, most preferably 550-610 mg based on 100 g dry weight of the composition.

In an embodiment when the composition is in the form of a oral nutritional supplement the daily amount of DHA administered is preferably 150 to 850 mg per day, more preferably 200 to 800 mg per day, most preferably 250 to 750 mg per day.

The present composition preferably comprises 30 to 240 mg DHA per 100 ml of the composition, preferably 35 to 220 mg DHA per 100 ml, more preferably 40 to 200 mg DHA per 100 ml composition.

Based on calories, preferably the nutritional composition according to the invention comprises 20 to 100 mg DHA per 100 kcal, preferably 25 to 90 mg DHA per 100 kcal, more preferably 30 to 80 mg DHA per 100 kcal based on the total energy content of the nutritional composition. The above numbers are based on the molar weight of DHA.

EPA is preferably administered in an amount of 40 to 350 mg, more preferably 50 to 300 mg, most preferably 60 to 250 mg EPA per day. In an embodiment when the composition is in the form of a tube feed, the daily amount of EPA administered is preferably 30 to 300 mg, more preferably 50 to 280 mg per day, most preferably 80 to 250 mg per day. The present composition preferably comprises in the range of 100-170 mg, more preferably 110-160 mg, most preferably 120-150 mg EPA based on 100 g dry weight of the composition.

In an embodiment, when the composition is in the form of an oral nutritional supplement, the daily amount of EPA administered is preferably 40 to 220 mg, more preferably 50 to 200 mg per day, most preferably 60 to 180 mg per day.

The present composition preferably comprises 5 to 60 mg, more preferably 7 to 55 mg, most preferably 9 to 50 mg EPA per 100 ml of the composition. Based on calories, preferably the nutritional composition according to the invention comprises 5 to 30 mg, more preferably 6 to 25 mg, most preferably 8 to 20 mg EPA per 100 kcal based on the total energy content of the nutritional composition. The above numbers are based on the molar weight of EPA. If EPA is present in the composition, the composition preferably comprises more DHA than EPA. i.e., the weight ratio of DHA to EPA is preferably more than 1, more preferably 1.5:1 to 4:1 even more preferably 2:1 to 4:1.

In terms of daily dosage, the present use and method preferably comprises the administration of preferably comprises 200 to 2000 mg omega-3 fatty acids (preferably DHA+EPA+DPA, most preferably DHA+EPA) per day, more preferably 230 to 1750 mg per day, even more preferably 250 to 1600 mg EPA per day. The present composition preferably comprises 0.3 to 3 wt % omega-3 fatty acids (more preferably DHA+EPA+DPA, most preferably DHA+EPA) on the total fatty acids, more preferably 0.5 to 2.5 wt %, even more preferably 0.75 to 2.0 wt % omega-3 fatty acids based on total fatty acids.

The present composition preferably comprises 0.1 to 3.5 wt % DHA based on total fatty acids, preferably 0.3 to 3 wt % DHA based on total fatty acids, more preferably 0.5 to 2.5 wt % DHA based on total fatty acids. The present composition preferably comprises between 0.05 to 0.30 wt % EPA based on total fatty acids, more preferably 0.1 to 0.25 wt %, even more preferably 0.15 to 0.20 wt % EPA based on total fatty acids.

The above-mentioned ratios and amounts take into account and optimize several aspects, including taste (too high n-3 LC-PUFA levels reduce taste, resulting in a reduced user compliance), balance between DHA and precursors thereof to ensure optimal effectiveness while maintaining low-volume formulations.

### Vitamin B2 and Further B Vitamins

The present composition according to the invention comprises vitamin B2. In an embodiment the composition may comprise a combination of DHA, vitamin B2, vitamin D3 in combination with EPA and one or more of choline and/or PC; and dietary fibres and/or dietary butyrate. In a further embodiment the composition may comprise a combination of omega-3 fatty acids DHA and EPA, vitamin B2, vitamin D3, choline and one or more of dietary fibres and dietary butyrate.

In some embodiments, the composition may comprise further B vitamins, preferably the composition comprises at least two B vitamins, wherein one B vitamin is B2 and the second B vitamin is selected from the group of vitamin B1 (thiamine), B3 (niacin or niacinamide), vitamin B5 (pantothenic acid), B6 (pyridoxine, pyridoxal, or pyridoxamine, or pyridoxine hydrochloride), vitamin B7 (biotin) vitamin B9 (folic acid or folate), and vitamin B12 (cobalamins). Functional equivalents are encompassed within these terms. The term “vitamin B12” incorporates all cobalamin equivalents known in the art. B-vitamins are known as nutritional co-factors that act as biochemical “spark plugs” in mitochondria, and therefore act to further nurture and support optimal neural metabolism. In an embodiment the composition comprises DHA, vitamin D and vitamin B2. In a further embodiment the composition comprises DHA, vitamin D, vitamin B2 and optionally together with vitamin B6 and/or vitamin B9.

Preferably, B vitamin in the context of the composition according to the invention comprises at least three B vitamins, wherein the first B vitamin is B2 and the second and third B vitamin are selected from the group of vitamin B1, B3, B5, B6, B7, vitamin B12 and vitamin B9. Preferably the composition comprises vitamin B2, B6 and B9, more preferably vitamin B2, B6, B9 and B12, even more preferably vitamin B1, vitamin B2, vitamin B6, vitamin B9 and vitamin B12. In one embodiment the composition according to the invention may include all of vitamin B1 (thiamine), B2 (riboflavin), B6 (pyridoxine, pyridoxal, or pyridoxamine, or pyridoxine hydrochloride), vitamin B9 (folic acid or folate), and vitamin B12 (cyanocobalamin). In some embodiment the composition may comprise further B vitamins, further B vitamins as used herein are vitamin B1, B3, B5 and B7 which the composition may comprise in addition to vitamin B2 and one or more of vitamin B6 and vitamin B9.

If present in the composition, the vitamin B1 is preferably present in an amount to provide a daily dosage in the range of 0.7 to 5 mg, preferably in the range of 0.8 to 4.9 mg, more preferably in the range of 0.9 to 4.8 mg. When the composition is in the form of a tube feed the daily amount of vitamin B1 administered is preferably 1.2 to 5.5 mg per day, more preferably 1.5 to 5 mg per day, most preferably 1.6 to 4.8 mg per day. When the composition is in the form of a oral nutritional supplement the daily amount of vitamin B1 administered is preferably 0.7 to 2.9 mg per day, more preferably 0.8 to 2.8 mg per day, most preferably 0.9 to 2.7 mg per day. If present, the composition preferably comprises 0.1 to 0.9 mg vitamin B1 per 100 ml, more preferably 0.15 to 0.8 mg vitamin B1 per 100 ml, more preferably 0.2 to 0.75 mg vitamin B1 per 100 ml. Based on calories, preferably the nutritional composition according to the invention may comprise 0.05 to 0.4 mg vitamin B1 per 100 kcal, preferably 0.1 to 0.35 mg vitamin B1 per 100 kcal, more preferably 0.15 to 0.30 vitamin B1 per 100 kcal based on the total energy content of the composition. The above numbers are based on the molar weight of thiamin.

In a preferred embodiment, the vitamin B1 is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. The RDI is a common reference in the art, for example herewith is referred to the population reference intake (PRI) such as according to the Summary of dietary reference values for the EU population as derived by the European Food Safety Authority (EFSA) as determined in September 2017. In the EU the RDI based on the ESFA for vitamin B1 is 0.1 mg per 238.83 calories intake.

In the composition according to the invention, the vitamin B2 is preferably present in an amount to provide a daily dosage in the range of 0.8 to 7.6 mg, preferably in the range of 1 to 7.4 mg, more preferably in the range of 1.2 to 7.2 mg per day, most preferably in the range of 1.4 to 7.2 mg per day. The present composition preferably comprises vitamin B2 in an amount in the range of 1.23-1.53 mg, more preferably 1.27-1.49 mg, most preferably 1.31-1.44 mg based on 100 g dry weight of the composition.

When the composition is in the form of a tube feed, the daily amount of vitamin B2 administered is preferably 2 to 7.6 mg per day, more preferably 2.2 to 7.4 mg per day, most preferably 2.4 to 7.2 mg per day. When the composition is in the form of a oral nutritional supplement the daily amount of vitamin B2 administered is preferably 0.8 to 4 mg per day, more preferably 1 to 3.8 mg per day, even more preferably 1.2 to 3.6 mg per day and most preferably 1.4 to 3.6 mg per day. The composition preferably comprises 0.1-1.4 mg vitamin B2 per 100 ml, more preferably 0.2-1.2 mg vitamin B2 per 100 ml, more preferably 0.3-1 mg vitamin B2 per 100 ml. Based on calories, preferably the nutritional composition according to the invention may comprise 0.4 to 0.6 mg vitamin B2 per 100 kcal, preferably 0.15 to 0.5 mg vitamin B2 per 100 kcal, more preferably 0.2 to 0.4 mg vitamin B2 per 100 kcal based on the total energy content of the composition. The above numbers are based on the molar weight of riboflavin.

In a further preferred embodiment, vitamin B2 is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. The RDI is a common reference in the art, for example herewith is referred to the population reference intake (PRI) such as according to the Summary of dietary reference values for the EU population as derived by the EFSA as determined in September 2017. In the EU the RDI based on the ESFA for vitamin B2 is 1.6 mg per day.

If present in the composition, the vitamin B3 is preferably present in an amount to provide a daily dosage in the range of 3 to 45 mg, preferably in the range of 3.5 to 42 mg, more preferably in the range of 3.8 to 40 mg niacin equivalent per day. When the composition is in the form of a tube feed the daily amount of vitamin B3 administered is preferably 11 to 42 mg per day, more preferably 12 to 40 mg per day, most preferably 12.5 to 39 mg niacin equivalents per day. When the composition is in the form of a oral nutritional supplement the daily amount of vitamin B3 administered is preferably 3.4 to 20 mg per day, more preferably 3.6 to 19 mg per day, most preferably 3.8 to 18 mg niacin equivalents per day. If present, the composition preferably comprises 1.2 to 6 mg niacin equivalent per 100 ml, more preferably 1.4 to 5.5 mg niacin equivalent per 100 ml, more preferably 1.6 to 5 mg niacin equivalent per 100 ml. Based on calories, preferably the nutritional composition according to the invention may comprise 1 to 2.5 mg niacin equivalent per 100 kcal, preferably 1.1 to 2.2 mg niacin equivalent per 100 kcal, more preferably 1.2 to 2 mg niacin equivalent per 100 kcal based on the total energy content of the composition. The above numbers are based on the molar weight of niacin.

If present in the composition, the vitamin B5 is preferably present in an amount to provide a daily dosage in the range of 0.8 to 15 mg, preferably in the range of 1 to 14 mg, more preferably in the range of 1.2 to 13 mg per day. When the composition is in the form of a tube feed the daily amount of vitamin B5 administered is preferably 3.5 to 14 mg per day, more preferably 4 to 13 mg per day, most preferably 4.1 to 12.6 mg per day. When the composition is in the form of a oral nutritional supplement the daily amount of vitamin B5 administered is preferably 0.8 to 7.5 mg per day, more preferably 1 to 7 mg per day, most preferably 1.2 to 6.8 per day. If present, the composition preferably comprises 0.3 to 2.2 mg vitamin B5 per 100 ml, more preferably 0.4 to 2 mg vitamin B5 per 100 ml, more preferably 0.5 to 1.8 mg vitamin B5 per 100 ml. Based on calories, preferably the nutritional composition according to the invention may comprise 0.2 to 1 mg vitamin B5 per 100 kcal, preferably 0.3 to 0.9 mg vitamin B5 per 100 kcal, more preferably 0.4 to 0.8 mg vitamin B5 per 100 kcal based on the total energy content of the composition. The above numbers are based on the molar weight of panthothenic acid.

The active form of vitamin B6, pyridoxal 5′-phosphate (PLP), serves as a co-enzyme in multiple enzyme reactions in amino acid, glucose, and lipid metabolism and is known to be the rate limiting factor in the synthesis of some neurotransmitters.

If present in the composition, the vitamin B6 is preferably present in an amount to provide a daily dosage in the range of 0.7 to 9 mg, preferably in the range of 0.8 to 8 mg, more preferably in the range of 0.9 to 7.5 mg. When the composition is in the form of a tube feed the daily amount of vitamin B6 administered is preferably 1.5 to 9 mg per day, more preferably 2 to 8 mg per day, most preferably 2.5 to 7.5 mg per day. When the composition is in the form of a oral nutritional supplement the daily amount of vitamin B6 administered is preferably 0.7 to 3.5 mg per day, more preferably 0.8 to 3 mg per day, most preferably 0.9 to 2.7 mg per day. If present, the composition preferably comprises 0.2 to 1 mg vitamin B6 per 100 ml, more preferably 0.25-0.8 mg vitamin B6 per 100 ml, more preferably 0.3 to 0.7 mg vitamin B6 per 100 ml. Based on calories, preferably the composition according to the invention may comprise 0.1 to 0.5 mg vitamin B6 per 100 kcal, preferably 0.2 to 0.4 mg vitamin B6 per 100 kcal, more preferably 0.25 to 0.3 mg vitamin B6 per 100 kcal based on the total energy content of the composition. The above numbers are based on the molar weight of pyridoxine.

In a preferred embodiment, the vitamin B6 is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin B6 is 1.65 mg per day.

If present in the composition, the vitamin B7 is preferably present in an amount to provide a daily dosage in the range of 8 to 95 μg, preferably in the range of 9 to 90 μg, more preferably in the range of 9.5 to 85 μg per day. When the composition is in the form of a tube feed the daily amount of vitamin B7 administered is preferably 24 to 95 μg per day, more preferably 26 to 90 μg per day, most preferably 28 to 88 μg per day. When the composition is in the form of a oral nutritional supplement the daily amount of vitamin B7 administered is preferably 9.4 to 45 μg per day, more preferably 9.6 to 42 μg per day, most preferably 9.8 to 41 μg per day. If present, the composition preferably comprises 2 to 14 μg vitamin B7 per 100 ml, more preferably 3 to 12 μg vitamin B7 per 100 ml, more preferably 3.5 to 11 μg vitamin B7 per 100 ml. Based on calories, preferably the composition according to the invention may comprise 1.5 to 6 μg vitamin B7 per 100 kcal, preferably 2 to 5.5 μg vitamin B7 per 100 kcal, more preferably 2.5 to 5 μg vitamin B7 per 100 kcal based on the total energy content of the composition. The above numbers are based on the molar weight of biotin.

Vitamin B9 or folic acid is an essential nutrient that is converted by the body into folate, which is required for DNA and RNA biosynthesis, DNA repair and one-carbon metabolic reactions. Decreased folate levels have frequently been reported in patients suffering from neurodegenerative diseases (Kronenberg, G, Curr Mol Med, 2009).

If present in the composition, the vitamin B9 is preferably present in an amount to provide a daily dosage in the range of 150 to 900 μg, preferably in the range of 175 to 800 μg, more preferably in the range of 200 to 750 μg per day. When the composition is in the form of a tube feed the daily amount of vitamin B9 administered is preferably 200 to 800 μg per day, more preferably 225 to 775 μg per day, most preferably 240 to 750 μg per day. When the composition is in the form of a oral nutritional supplement the daily amount of vitamin B9 administered is preferably 150 to 700 μg per day, more preferably 175 to 650 μg per day, most preferably 200 to 600 μg per day. If present, the composition preferably comprises 20 to 225 μg vitamin B9 per 100 ml, more preferably 25 to 200 μg vitamin B9 per 100 ml, more preferably 30 to 175 μg vitamin B9 per 100 ml. Based on calories, preferably the composition according to the invention may comprise 16 to 80 μg vitamin B9 per 100 kcal, preferably 18 to 75 μg vitamin B9 per 100 kcal, more preferably 20 to 70 μg vitamin B9 per 100 kcal based on the total energy content of the composition. The above numbers are based on the molar weight of folic acid.

In a preferred embodiment, the vitamin B9 is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin B9 is 330 μg dietary Folate Equivalents per day.

If present in the composition, the vitamin B12 is preferably present in an amount to provide a daily dosage in the range of 2 to 20 μg, preferably in the range of 2.5 to 19 μg, more preferably in the range of 3 to 18 μg per day. When the composition is in the form of a tube feed the daily amount of vitamin B9 administered is preferably 4 to 22 μg per day, more preferably 5 to 20 μg per day, most preferably 6 to 18 μg per day. When the composition is in the form of a oral nutritional supplement the daily amount of vitamin B9 administered is preferably 2 to 12 μg per day, more preferably 2.5 to 10 μg per day, most preferably 3 to 9 μg per day. If present, the composition preferably comprises 0.5 to 3 μg vitamin B12 per 100 ml, more preferably 0.6 to 2.6 μg vitamin B12 per 100 ml, more preferably 0.7 to 2.4 μg vitamin B12 per 100 ml. Based on calories, preferably the composition according to the invention may comprise 0.4 to 1.4 μg vitamin B12 per 100 kcal, preferably 0.5 to 1.2 μg vitamin B12 per 100 kcal, more preferably 0.6 to 1 μg vitamin B12 per 100 kcal based on the total energy content of the composition. The above numbers are based on the molar weight of cyanocobalamin.

In a preferred embodiment, the vitamin B12 is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin B12 is 4 μg per day.

### Vitamin D3

The present composition comprises vitamin D3. In an embodiment the composition may comprise a combination of DHA, vitamin B2, vitamin D3, in combination with EPA and one or more of choline and/or PC; and dietary fibres and/or dietary butyrate. In a further embodiment the composition according to the invention may comprise a combination of omega-3 fatty acids DHA and EPA, vitamin B2, vitamin D3, choline and one or more of dietary fibres and dietary butyrate.

Vitamin D is a group of fat-soluble secosteroids, the two major physiologically relevant forms of which are vitamin D2 (ergocalciferol) and vitamin D3 (cholecalciferol). These are known collectively as calciferol. Vitamin D without a subscript refers to all forms of vitamin D, either D1, D2, D3, or D4, in particular D2 and D3, or any mixture thereof. Vitamin D may be provided in an active (1,25(OH)₂D) or unactive (vitamin D3 or D2) form. The composition according to the invention comprises vitamin D3.

When ingested, vitamin D is hydroxylated in the liver (endoplasmic reticulum) to 25-hydroxycholecalciferol (25(OH)D), also known as calcidiol, by the enzyme 25-hydroxylase, produced by hepatocytes. Once made, the product is stored in the hepatocytes until it is needed and can be released into the plasma where it will be bound to an α-globulin. 25-hydroxycholecalciferol is then transported to the proximal tubules of the kidneys where it can be hydroxylated by one of two enzymes to different forms of vitamin D, one of which is active vitamin D (1,25(OH)₂D) and another which is inactive vitamin D (24,25(OH)D). The enzyme 1α-hydroxylase which is activated by parathyroid hormone (and additionally by low calcium or phosphate) forms the main biologically active vitamin D hormone with a C1 hydroxylation forming 1,25-dihydroxycholecalciferol (1,25(OH)₂D, also known as calcitriol). A separate enzyme hydroxylates the C24 atom forming 24R,25(OH)₂D3 when 1α-hydroxylase is not active, this inactivates the molecule from any biological activity.

The present composition preferably comprises 1.5 to 10 μg vitamin D3, preferably 2 to 9 μg vitamin D3 per 100 ml, more preferably 2.5 to 8 μg vitamin D3 per 100 ml composition. In the most preferred embodiment, the present composition comprises 10-15 μg vitamin D3 per 100 ml composition. According to one embodiment, vitamin D3 is provided in an amount of 5 to 80 μg per day, preferably 6 to 75 μg per day, more preferably 8 to 70 μg per day, even more preferably 10 to 65 μg per day. The present composition preferably comprises vitamin D3 in an amount in the range of 38-54 μg, more preferably 40-52 μg, most preferably 42-50 μg based on 100 g dry weight of the composition.

In an embodiment the composition is in the form of a tube feed and vitamin D3, is preferably provided in an amount of 14 to 80 μg per day, more preferably 16 to 75 μg per day, even more preferably 18 to 70 μg per day, most preferably 20 to 65 μg per day. In another embodiment when the composition is in the form of an oral nutritional supplement vitamin D3 is preferably provided in an amount of 5 to 45 μg per day, more preferably 6 to 40 μg per day, even more preferably 8 to 35 μg per day, most preferably 10 to 30 μg per day.

Based on calories, preferably the nutritional composition according to the invention comprises 1.3 to 4.5 μg vitamin D3, per 100 kcal, preferably 1.5 to 4 μg vitamin D3 per 100 kcal, more preferably 2 to 3.5 μg per 100 kcal based on the total energy content of the nutritional composition. The above numbers are based on the molar weight of vitamin D3.

In a further preferred embodiment, vitamin D3 is preferably present in a therapeutic effective amount if the nutritional supplement or composition provides at least 1.2, preferably at least 1.3 times the recommended daily intake (RDI), and preferably not more than 7 times the RDI. In a preferred aspect from 1.2 to 5 times, preferably 1.3 to 4.5 times, more preferably 1.4 to 4 times the recommended daily intake is provided. The RDI is a common reference in the art, for example herewith is referred to the population reference intake (PRI) such as according to the Summary of dietary reference values for the EU population as derived by the EFSA as determined in September 2017. In embodiments, the RDI of vitamin D3 is 15 μg per day.

### Choline

The present composition preferably further comprises choline. Choline may be present as such, or in the form of a choline salt and/or choline ester. The choline salt is preferably selected from choline chloride, choline bitartrate, choline butyrate or choline stearate. The choline ester is preferably selected from a phosphatidylcholine and lyso-phosphatidyl choline, preferably phosphatidylcholine. In a preferred embodiment the present composition comprises both a) choline as such or a choline salt and b) a choline ester, preferably phosphatidylcholine.

The present composition for use and method preferably comprises the administration of more than 70 mg of a) choline or choline salt per day, preferably 70 to 1300 mg per day, more preferably 80 to 1200 mg per day, most preferably 100 to 1100 mg per day. In an embodiment when the composition is in the form of a tube feed the daily amount of choline or choline salt administered is preferably 200 to 1400 mg per day, more preferably 300 to 1200 mg per day, most preferably 350 to 1100 mg per day. In another embodiment when the composition is in the form of a oral nutritional supplement the daily amount of choline or choline salt administered is preferably 70 to 500 mg per day, more preferably 80 to 400 mg per day, most preferably 100 to 300 mg per day.

The present composition preferably comprises 30 to 180 mg choline or choline salts per 100 ml of the liquid composition, preferably 35 mg to 170 mg choline per 100 ml, more preferably 40 to 160 mg choline per 100 ml composition, most preferably 45 mg to 150 mg choline per 100 ml. Based on calories, preferably the nutritional composition according to the invention comprises 25 to 110 mg choline per 100 kcal, preferably 30 to 105 mg choline per 100 kcal, more preferably 33 to 100 mg per 100 kcal based on the total energy content of the nutritional composition.

The above numbers are based on choline, the amounts of choline salts or esters can be calculated taking the molar equivalent to choline into account. In a preferred embodiment, the present composition comprises in the range of 5-15 mg choline provided in the form of phosphatidylcholine, more preferably 6-12 mg, most preferably 7-10 mg based on 100 g dry weight of the composition. Herein and throughout the description is meant that the weight of choline provided in the form of a choline ester, preferably phosphatidylcholine, is the weight of choline as present in the choline ester, preferably phosphatidylcholine.

Choline is necessary for the synthesis of acetylcholine, which latter is critical for the communication between neurons in the brain and plays a role in cognitive processes such as attention, concentration, and memory formation. Choline is a component of phospholipids, which are the building blocks of cell membranes and adequate choline intake ensures the integrity and fluidity of neuronal membranes, which is essential for the proper functioning of neurons. Choline deficiency can lead to impaired membrane function and affect neuronal signaling.

Furthermore, choline is involved in the process of neurotransmitter release in neurons. It helps regulate the release of neurotransmitters, including acetylcholine, by maintaining the balance of acetylcholine within the synaptic vesicles. Proper neurotransmitter release is crucial for neuronal communication and overall brain function. Also, choline is involved in neurogenesis, the process of generating new neurons in the brain. It supports the growth and development of neuronal cells. Finally, choline has been linked to memory enhancement and cognitive function. It has been suggested that choline supplementation may improve memory and attention in certain populations, particularly older adults (Liu, L, Behavioural Neurology, 2021).

The present composition may comprise choline in the form of a choline ester, preferably provided in the form of phospholipids. Preferably, one or more phospholipid(s) is/are present in the composition according to the invention. The one or more phospholipid(s) is/are selected from the group consisting of phosphatidic acid (PA), phosphatidylethanolamine (PE), phosphatidylcholine (PC), phosphatidylserine (PS) and phosphoinositides (PI), preferably at least phosphatidyl choline. Phospholipids are in a preferred embodiment a source of the choline ester phosphatidylcholine.

Suitable sources of phospholipids herein are soya lecithin, egg lecithin as well as rapeseed and sunflower lecithin. Milk contains phospholipids in the milk fat globule membrane (MFGM), suitable sources include milk phospholipid concentrate such as those commercially available from Lecico GmbH, whey protein concentrate Lacprodan® MFGM from Arla Foods. Depending on the source, phospholipids comprise about 10 to 40 wt % of phosphatidylcholine.

If present, the composition and use or method of use of the composition preferably comprises the administration of phospholipids in an amount of 0.6 to 6.6 g per day, preferably 0.8 to 6.4 g of phospholipids per day, more preferably 0.9 to 6.2 g of phospholipids per day, most preferably 1 to 6 g of phospholipids per day. In an aspect the amount of phospholipids is about 200 mg per day, preferably 0 to 350 mg phospholipids, more preferably 75 to 300 mg, even more preferably 90 to 250 mg phospholipids per day. The present composition preferably comprises 200-290 mg phospholipids, more preferably 210-280 mg, most preferably 220-265 mg phospholipids based on 100 g dry weight of the composition. The phospholipids preferably at least provide phosphatidylcholine. In a preferred embodiment, choline as provided in the form phosphatidylcholine is preferably in the range of 1 to 15 mg, preferably 2 to 12 mg, more preferably 3 to 10 mg per day.

If phospholipids are present, the present composition preferably comprises phospholipids in an amount of 150 and 900 mg per 100 ml, more preferably between 200 and 850 mg per 100 ml, most preferably 250 to 800 mg per 100 ml. In an alternative preferred embodiment, the present composition preferably comprises 20-200 mg phospholipids, more preferably 40-150 mg, most preferably 50-100 mg phospholipids per 100 ml of the composition. The phospholipids preferably at least provide phosphatidylcholine. In a preferred embodiment, choline as provided in the form of phosphatidylcholine is preferably in the range of 0.5-8 mg, more preferably 1-7 mg, most preferably 1.5-5 mg per 100 ml of the composition. In an embodiment, when the composition is in the form of a tube feed, the daily amount of phospholipids administered is preferably 1 to 7 g per day, more preferably 1.5 to 6.5 g per day, most preferably 2 to 6 g per day. In another embodiment when the composition is in the form of a oral nutritional supplement the daily amount of phospholipids administered is preferably 0.5 to 4 g per day, more preferably 0.75 to 3.5 g per day, most preferably 1 to 3 g per day.

Based on calories, preferably the nutritional composition according to the invention comprises 100 to 500 mg of phospholipids per 100 kcal, preferably 150 to 400 mg of phospholipids per 100 kcal, more preferably 200 to 350 mg of phospholipids per 100 kcal based on the total energy content of the nutritional composition. In an alternative preferred embodiment, the present composition preferably comprises 20-200 mg phospholipids, more preferably 40-150 mg, most preferably 50-100 mg phospholipids per 100 kcal based on total energy of the composition. The phospholipids preferably at least provide phosphatidylcholine. In a preferred embodiment, choline as provided in the form of phosphatidylcholine is preferably in the range of 0.5-8 mg, more preferably 1-7 mg, most preferably 1.5-5 mg per 100 kcal based on total energy of the composition. In a specific embodiment, the nutritional composition according to the invention, comprises a) choline or choline salts and b) phosphatidylcholine (PC). In such embodiment the weight ratio phosphatidylcholine to choline is usually more than 0.1, preferably more than 0.26, in particular 0.30-6, more preferably 0.36-3. Most preferably, the weight ratio phosphatidylcholine to choline is in the range of 40:60 to 60:40. Herein, the amount of choline in grams is to be calculated as the molar contribution of choline as provided by all choline sources (when orally digested and assuming 100% bioavailability, including PC), times the molecular weight of choline (104 g/mol). Herein, the molecular weight of PC is 810 gram/mol. So for example, including 400 mg choline chloride (having a molecular weight of 139.6 g/mol) and 200 mg phosphatidylcholine and 200 mg phospholipids other than PC would then result in a weight ratio of PC to choline of 200/[(104/139.6)×400+(104/810)×200]=200/[298+25.7]=0.62.

The presence of PC is in particular preferred since herewith the choline (salt) content can be reduced, whilst still providing a bioavailable choline source. Additionally dietary choline can be metabolized in the gut into trimethylamine (TMA) which in turn is oxidized in the liver into trimethylamine N-oxide (TMAO). Both TMA and TMAO have been associated with an increased risk of cardiovascular diseases (Zhu et al, Cell 2016; Jaworska et al. Cardiovac. Res 2019) and therefore it is preferred to provide choline both in free (salt) form and as phosphatidylcholine.

Preferably the composition according to the invention comprises the combination of DHA, vitamin B2, vitamin D3 and choline, optionally with one or more of dietary fibres and/or dietary butyrate. In a further preferred embodiment, the composition comprises a combination of omega-3 fatty acids DHA and EPA, vitamin B2, vitamin D3, choline and one or more of dietary fibres and/or dietary butyrate wherein choline is for at least 20 mole %, preferably at least 30 mole %, more preferably at least 40 mole % provided in the form of phosphatidylcholine, preferably the molar ratio choline (salts) and PC is in the range from 40:60 to 60:40, i.e, wherein 40 to 60 mole % choline is derived from phosphatidylcholine based on total choline in the composition. Preferably, the composition comprises a combination of omega-3 fatty acids DHA and EPA, vitamin B2, vitamin D3, choline and one or more of dietary fibres and dietary butyrate wherein choline is for at least 40 mole % provided in the form of phosphatidylcholine, preferably the molar ratio choline and PC is in the range from 40:60 to 60:40 and further comprises one of more selected from A, further B, C, and E vitamin(s), phospholipids, selenium and magnesium. As herein referred to, the molar ratio of choline and PC is referred to as the mole % choline derived from a choline salt and PC. More preferably the composition contains two or more of these further ingredients. The further ingredients and their respective amounts will be discussed hereinafter.

### Dietary Fibres and Dietary Butyrate

The composition according to the invention preferably comprises dietary fibres. In the context of the invention, dietary fibres for use in the composition are defined as dietary fibres or dietary fibre blends that produce butyrate, preferably they produce butyrate and propionate in a beneficial ratio.

In a preferred embodiment, the composition comprises a combination of DHA, vitamin B2, vitamin D3 in combination with EPA, dietary fibres and optionally one or more of choline and/or PC. In a further embodiment the composition according to the invention may comprise a combination of omega-3 fatty acids DHA and EPA, vitamin B2, vitamin D3, choline and one or more of dietary fibres, such as fibres selected from scGOS, IcFOS and low viscosity pectin, and may further comprise dietary butyrate. Herein, low viscosity pectin refers to a pectin that is partially hydrolyzed. In a preferred embodiment, the low viscosity pectin has a degree of polymerisation (DP) of less than 6000, and/or a molecular weight of less than 150 kDa.

The dietary fibres that may serve as a substrate for generating fatty acids, preferably produce acetate, butyrate, propionate, and hexanoate, more preferably propionate and butyrate in a beneficial ratio, allowing to increase the butyrate production of subjects.

The expression “a beneficial butyrate to propionate ratio” as used herein means that the weight of butyrate produced by fermentation of a fibre or mixture of fibres is at least equal or higher that the amount of propionate produced by the fermentation. For example, upon fermentation of locust bean gum or yeast beta-glucan more propionate than butyrate is produced, and these fibres have a butyrate to propionate ratio below 1 and cannot boost or increase butyrate production. In contrast, soy fibres and inulin are exemplary fibres that upon fermentation have a beneficial butyrate to propionate ratio.

In a preferred embodiment the fermentation of dietary fibres beneficially results in a fatty acid production wherein butyrate and propionate are produced in a weight ratio of butyrate:propionate at least 1, preferably at least 1.1. Fermentation profiles of dietary fibres concomitant butyrate to propionate ratio's from either a single source of fibres or blend of fibres, can be assessed using fermentation assays known in the art. Exemplary ways to assess the fermentation of fibres include using in fibres in fermentation models with faecal sample pools of healthy adults with exposure to approximately 10 mg/ml fibre for 24 hours at 37° C. under anaerobic conditions.

In a particular embodiment the use of fibre blends is preferred as dietary fibres. Exemplary fibre blends for use in the context of the present invention are blends that are fermentable and provide a beneficial C4:C3 weight ratio of at least 1, preferably at least 1.1, even more preferably at least 1.5. An exemplary fibre blend is a fibre mixture that comprises a) beta-galactooligosaccharides, b) inulin, c) resistant starch and/or d) soluble soy polysaccharides. The blend is fermentable and provides fatty acids, in particular at a beneficial C4:C3 weight ratio of at least 1, preferably at least 1.1, even more preferably at least 1.5.

A further exemplary fibre blend comprises a) FOS, b) inulin, c) soy fibre, d) resistant starch, e) acacia gum and/or f) cellulose. The blend is fermentable and provides fatty acids, in particular at a beneficial C4:C3 weight ratio of at least 1, preferably at least 1.1, even more preferably at least 1.5. A further exemplary fibre blend comprises a) arabinoxylan, b) oat beta-glucan, c) pectin, and/or d) resistant starch. The fibre mixture is fermentable and produces butyrate and propionate in a weight ratio of C4:C3 of at least 1, preferably at least 1.1, even more preferably at least 1.5.

A preferred fibre blend for use in the composition comprises a) scGOS, b) IcFOS and c) low-viscosity pectin. The fibre mixture is fermentable and produces butyric acid, in particular at a beneficial C4:C3 weight ratio of at least 1, preferably at least 1.1, even more preferably at least 1.5.

ScGOS refers to short chain GOS with a lower degree of polymerization, typically consisting of 2 to 6 galactose units. Examples of scGOS include but are not limited to galactobiose, lactulose, isomaltooligosaccharide. In a preferred embodiment, the scGOS comprises galacto-oligosaccharide with beta-(1,4), beta-(1,3) and/or beta-(1,6) glycosidic bonds and a terminal glucose. Transgalacto-oligosaccharide is for example available under the trade name Vivinal® GOS (Domo FrieslandCampina Ingredients), Bi2muno (Clasado), Cup-oligo (Nissin Sugar) and Oligomate55 (Yakult).

LcFOS refers to long chain FOS with a higher degree of polymerization, preferably consisting of a degree of polymerization above 8, more preferably between 10 and 100, most preferably between 20 and 50. Examples of IcFOS include but are not limited to inulin and oligofructose. LcFOS may refer to oligosaccharides comprising beat-linked fructose units, which are preferably linked by beta-(2,1) and/or beta-(2,6) glycosidic linkages. Preferably, the IcFOS contains a terminal beta-(2,1) glycosidic linked glucose. In a further preferred embodiment inulin is used. Inulin is a type of IcFOS wherein at least 75% of the glycosidic linkages are beta-(2,1) linkages. Typically, inulin has an average chain length between 8 and 60 monosaccharide units. A suitable IcFOS for use in the compositions according to the method or use of the present invention is commercially available under the trade name Raftiline® HP (Orafti). Other suitable sources are Raftilose (Orafti), Fibrulose and Fibruline (Cosucra) and Frutafit and Frutalose (Sensus).

In one embodiment, the dietary fibres are a mixture of scGOS and IcFOS. Preferably the mixture of scGOS and IcFOS is present in a weight ratio of from 1/99 to 99/1, more preferably from 1/19 to 19/1, more preferably from 1/1 to 19/1, more preferably from 2/1 to 15/1, more preferably from 5/1 to 12/1, even more preferably from 8/1 to 10/1, even more preferably in a ratio of about 9/1.

In a preferred embodiment, another dietary fibre is low-viscosity pectin. Low-viscosity pectin is a modified form of pectin, a naturally occurring polysaccharide found in the cell walls of fruits and vegetables. Low-viscosity pectin is specifically processed to have a lower molecular weight and a reduced ability to form gels compared to regular pectin. The modification of pectin to create low-viscosity pectin involves various methods such as acid hydrolysis or enzymatic treatment. These processes break down the long chains of pectin molecules into shorter chains, resulting in a lower viscosity and improved dispersibility in liquid formulations.

In a further preferred embodiment, the composition comprises a mixture of dietary fibres comprising scGOS, IcFOS and low viscosity pectin, wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5.

Preferably the daily dosage comprises 2.5 to 12.5 g, more preferably 3 to 12 g, most preferably 3.5 to 11.5 g dietary fibres. In another preferred embodiment, the composition comprises 0.5 to 4.0 g, more preferably 1.0 to 3.5 g, most preferably 1.5 to 3.0 g dietary fibres per 100 ml of the composition. In another preferred embodiment, the composition comprises per 100 kcal 0.75 to 2.5 g, more preferably 1.0 to 2.0 g, most preferably 1.1 to 1.5 g dietary fibres. The present composition preferably comprises dietary fibres in the range of 9.0-11.3 g, more preferably 9.2-11 g, most preferably 9.8-10.8 g based on 100 g dry weight of the composition.

The present composition according to the invention preferably comprises dietary butyrate. In the context of the invention, dietary butyrate for use in the composition is defined as butyric acid, butyrate salts, butyrate esters such as preferably glyceride bound forms of butyrate comprising one, two or three butyrate chains.

The dietary butyrate can be supplied by any suitable source known in the art. Non-limiting sources of dietary butyrate includes animal source fats and derived products, such as but not limited to milk, milk fat, butter fat, butter oil, butter, buttermilk, butter serum, cream; microbial fermentation derived products, such as but not limited to yogurt and fermented buttermilk; and plant source derived seed oil products, such as pineapple and/or pineapple oil, apricot and/or apricot oil, barley, oats, brown rice, bran, green beans, legumes, leafy greens, apples, kiwi, oranges. In some embodiments, the dietary butyrate is synthetically produced but preferably the dietary butyrate is supplied by a natural source as defined here above.

In some embodiments, the composition comprises the dietary butyric acid in the form of butyrate salts, for example, sodium butyrate, potassium butyrate, calcium butyrate, magnesium butyrate, choline butyrate, and combinations thereof. In certain embodiments, dietary butyric acid comprises a suitable butyrate salt that has been coated with one or more fats or lipids. Preferably the dietary butyric acid is supplied as a butyrate ester, such as in a preferred embodiment as part of a (tri)glyceride. This is advantageous because butyric acid is volatile (and malodorous) when provided in free or salt form. In (tri)glyceride form the butyrate will be released in and after the stomach due to the action of lipases.

In a preferred embodiment, dietary butyrate for use in the composition comprises a butyrate chain-containing glyceride, preferably a butyrate chain-containing triglyceride. In the context of the invention, the butyrate chain-containing glyceride comprises at least one butyrate chain, preferably at least two or three butyrate chains. Preferably dietary butyrate comprises tributyrin (i.e., triglyceride with 3 butyric acid chains attached to the glycerol backbone via ester bonds). If the butyrate is added as a triglyceride or diglyceride, it may be that 0, 1, or 2 of the other side chains are other fatty acid chains that butyric acid, or in other words, the diglycerides or triglycerides may be mixed glycerides wherein at least one of the fatty acid chains is butyrate.

Preferably, the daily dosage comprises 0.125 g to 0.65 g dietary butyrate, more preferably 0.025 g to 0.65 g, most preferably 0.043 g to 0.65 g dietary butyrate based on weight of butyric acid.

In another preferred embodiment, the composition comprises 0.01 to 0.175 g dietary butyrate per 100 ml, more preferably 0.02 to 0.175 g dietary butyrate per 100 ml, and most preferably 0.035 to 0.175 g dietary butyrate per 100 ml, based on weight of butyric acid. In another preferred embodiment, the composition comprises 0.015 to 0.25 g dietary butyrate per 100 kcal, more preferably 0.03 to 0.25 g dietary butyrate per 100 kcal, and most preferably 0.05 to 0.25 g dietary butyrate per 100 kcal, based on weight of butyric acid.

In a further preferred embodiment, the composition according to the invention comprises a mixture of dietary fibres and dietary butyrate. Preferably the mixture of dietary fibres and dietary butyrate contains at least 50 wt % of dietary fibres, more preferably the weight ratio dietary fibres to dietary butyrate in the mixture is in the range from 80:20 to 50:50, more preferably 75:25 to 60:40.

Neurons have high energy demands, and butyrate as can be derived from dietary fibres, and dietary butyrate can serve as an alternative energy source for neurons. It can be metabolized by neurons to produce ATP (adenosine triphosphate), which is the primary energy currency of cells. Also, butyrate can exhibit neuroprotective effects by reducing oxidative stress and inflammation in neurons. It can help protect neurons from damage caused by harmful molecules and promote their survival. Importantly, butyrate has been found to increase the production of brain-derived neurotrophic factor (BDNF) in neurons. BDNF is a protein that plays a crucial role in neuronal growth, survival, and synaptic plasticity, which is important for learning and memory. Finally, butyrate possesses anti-inflammatory properties that can help modulate the inflammatory response in neurons. Excessive inflammation in the brain can be detrimental to neuronal health, and butyrate's anti-inflammatory effects may contribute to neuroprotection.

### Vitamin A

The present composition may further comprise vitamin A. Vitamin A is a fat-soluble vitamin which comes in several forms (as retinol, retinal, retinoic acid or retinyl ester). Any functional form of vitamin A known in the art is suitable to be used herein, including retinol (in particular retinol esters), retinal, retinoic acid, beta-carotene, provitamin A, or any combination thereof. Preferably, the composition comprises retinol, in particular retinyl acetate and/or retinyl palmitate.

Vitamin A intakes or requirements are generally expressed in terms of retinol equivalents (RE). One RE is defined as the biological activity associated with 1 μg of all-trans retinol. Hence 1 μg Retinol Equivalent is similar to 1 μg of all-trans retinol. 1 International Unit (IU) retinol corresponds to 0.3 μg Retinol Equivalents.

If present in the composition, vitamin A is preferably administered in an amount of 300 to 2800 μg Retinol Equivalents per day, more preferably 350 to 2700 μg Retinol Equivalents per day, even more preferably 650 to 900 μg Retinol Equivalents per day, most preferably 375 to 2600 μg Retinol Equivalents per day. In a preferred embodiment, the vitamin A is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin A is 700 μg Retinol Equivalents per day. In an embodiment the composition is in the form of a tube feed and the daily amount of vitamin A administered is preferably 700 to 2850 μg Retinol Equivalents per day, more preferably 800 to 2750 μg Retinol Equivalents per day, most preferably 850 to 2600 μg Retinol Equivalents per day.

In another embodiment the composition is in the form of a oral nutritional supplement and the daily amount of vitamin A administered is preferably 300 to 1200 μg Retinol Equivalents per day, more preferably 350 to 1150 μg Retinol Equivalents per day, most preferably 375 to 1125 μg Retinol Equivalents per day. The present composition may preferably comprise vitamin A, preferably 50 to 400 μg Retinol Equivalents per 100 ml of the composition, more preferably 75 to 350 μg Retinol Equivalents per 100 ml, most preferably 100 to 300 μg Retinol Equivalents per 100 ml composition.

Based on calories, preferably the nutritional composition according to the invention may comprise 60 to 150 μg Retinol Equivalents per 100 kcal, preferably 70 to 140 μg Retinol Equivalents per 100 kcal, more preferably 80 to 130 μg Retinol Equivalents per 100 kcal based on the total energy content of the nutritional composition.

### Vitamin C

The present composition may further comprise vitamin C. Vitamin C is an antioxidant that may provide beneficial characteristics in neurodegenerative diseases and after neurotrauma and brain injury and/or brain damage. Vitamin C includes functional equivalents thereof including sodium ascorbate and calcium ascorbate, and may be present in an amount to provide a daily dosage in the range of 75 to 700 mg, preferably in the range of 85 to 600 mg, more preferably in the range of 95 to 550 mg per day. When the composition is in the form of a tube feed the daily amount of vitamin C administered is preferably 140 to 600 mg per day, more preferably 160 to 575 mg per day, most preferably 180 to 550 mg per day. When the composition is in the form of a oral nutritional supplement the daily amount of vitamin C administered is preferably 80 to 320 mg per day, more preferably 90 to 300 mg per day, most preferably 95 to 290 mg per day. In one embodiment, if present vitamin C is present in an amount in the range of 10 to 100 mg, preferably in the range of 15 to 90 mg, more preferably in the range of 20 to 80 mg per 100 ml of the composition. Based on calories, preferably the composition according to the invention may comprise 12 to 40 mg vitamin C per 100 kcal, preferably 14 to 35 mg vitamin C per 100 kcal, more preferably 16 to 32 mg vitamin C per 100 kcal based on the total energy content of the composition. The above numbers are based on the molar weight of ascorbic acid.

In a preferred embodiment, the vitamin C is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin C is 102.5 mg per day.

### Vitamin E

The composition according to the invention may comprise vitamin E. Vitamin E refers to compounds having vitamin E activity as known in the art, typically tocopherol and/or an equivalent thereof.

If included, vitamin E may be present in the composition in an amount to provide a daily dosage in the range of 8 to 75 mg, preferably in the range of 10 to 70 mg, more preferably in the range of 12.5 to 70 mg alpha-TE. When the composition is in the form of a tube feed the daily amount of vitamin E administered is preferably 18 to 80 mg per day, more preferably 20 to 75 mg per day, most preferably 22 to 70 mg alpha-TE per day. When the composition is in the form of a oral nutritional supplement the daily amount of vitamin E administered is preferably 8 to 42 mg per day, more preferably 10 to 40 mg per day, most preferably 12.5 to 37.5 mg alpha-TE per day. Such amounts of vitamin E prevent oxidative damage to the injury site. In one embodiment, tocopherol and/or equivalent is present in an amount in the range of 2 to 14 mg alpha-TE, preferably in the range of 2.5 to 12 mg alpha-TE, more preferably in the range of 2.75 to 10 mg alpha-TE per 100 ml of the composition. Based on calories, preferably the composition according to the invention may comprise 1 to 5.5 mg alpha-TE per 100 kcal, preferably 1.5 to 5 mg alpha-TE per 100 kcal, more preferably 2 to 4.5 mg alpha-TE per 100 kcal based on the total energy content of the composition.

In a preferred embodiment, the vitamin E is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for vitamin E is 12 mg per day. The term “tocopherol and/or an equivalent thereof”, as used in this description, comprises tocopherols (e.g. alpha- and gamma-), tocotrienols, pharmaceutical and/or nutritional acceptable derivatives thereof and any combination thereof. The above numbers are based on alpha-tocopherol equivalents (alpha-TE) and the molecular weight thereof, as recognized in the art.

### Vitamin K1

The composition according to the invention may optionally comprise vitamin K, preferably vitamin K1, although it is preferred that the composition does not comprise or comprise minimal amounts of vitamin K. Vitamin K is a general name for a group of compounds containing a 2-methyl-1,4-naphthoquinone nucleus with different lipophilic side chains at position 3, in the following also referred to as “the K-vitamins”. Vitamin K naturally occurs in the form of vitamin K1 (phytomenadione, phytonadione, α-phylloquinone) and vitamin K2 (menaquinone) which is produced by gastrointestinal bacteria in basically two different sub-types, namely vitamin K2(35) (C46H64O2) and vitamin K2(30) (C41H56O2). Vitamin K3 (menadione) is a pro-vitamin which can be converted to vitamin K2 by animals.

If present in the composition, the vitamin K1 is preferably present in a daily dosage at most 240 μg, preferably at most 200 μg, more preferably at most 100 μg per day. The present composition preferably comprises less than 40 μg vitamin K1 per 100 ml, more preferably less than 35 μg vitamin K1 per 100 ml, more preferably less than 30 μg vitamin K1 per 100 ml. Based on calories, preferably the composition according to the invention may comprise less than 18 μg vitamin K1 per 100 kcal, preferably less than 16 μg vitamin K1 per 100 kcal, more preferably less than 15 μg vitamin K1 per 100 kcal based on the total energy content of the composition. The above numbers are based on the molar weight of phylloquinone. In a preferred embodiment the composition comprises less than 15 μg vitamin K1 per 100 kcal.

### Selenium

The present composition may comprise selenium. The antioxidant activity of selenium may advantageously prevent and/or inhibit damages to brain areas.

If present in the composition, selenium is preferably present in an amount to provide a daily dosage in the range of 30 to 275 μg, preferably in the range of 35 to 250 μg, more preferably in the range of 38 to 225 μg per day. When the composition is in the form of a tube feed the daily amount of selenium administered is preferably 60 to 275 μg per day, more preferably 70 to 250 μg per day, most preferably 75 to 225 μg per day. When the composition is in the form of a oral nutritional supplement the daily amount of selenium administered is preferably 25 to 140 μg per day, more preferably 30 to 130 μg per day, most preferably 35 to 120 μg per day. The present composition preferably comprises 8 to 45 μg selenium per 100 ml, more preferably 10 to 40 μg selenium per 100 ml, more preferably 11 to 35 μg selenium per 100 ml. Based on calories, preferably the composition according to the invention may comprise 4 to 18 μg selenium per 100 kcal, preferably 6 to 16 μg selenium per 100 kcal, more preferably 8 to 14 μg selenium per 100 kcal based on the total energy content of the composition.

In a preferred embodiment, the selenium is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for selenium is 70 μg per day.

### Magnesium

The present composition may comprise magnesium. Magnesium plays an essential role in nerve transmission and neuromuscular conduction. It also functions in a protective role against excessive excitation that can lead to neuronal cell death (excitotoxicity).

If present in the composition, magnesium is preferably present in an amount to provide a daily dosage in the range of 125 to 850 mg, preferably in the range of 150 to 800 mg, more preferably in the range of 170 to 750 mg per day. When the composition is in the form of a tube feed the daily amount of magnesium administered is preferably 200 to 780 mg per day, more preferably 220 to 760 mg per day, most preferably 240 to 740 mg per day. When the composition is in the form of a oral nutritional supplement the daily amount of magnesium administered is preferably 150 to 600 mg per day, more preferably 160 to 550 mg per day, most preferably 170 to 525 mg per day. The present composition preferably comprises 20 to 160 mg magnesium per 100 ml, more preferably 25 to 150 mg magnesium per 100 ml, more preferably 30 to 140 mg magnesium per 100 ml. Based on calories, preferably the composition according to the invention may comprise 10 to 70 mg magnesium per 100 kcal, preferably 15 to 65 magnesium per 100 kcal, more preferably 20 to 60 mg magnesium per 100 kcal based on the total energy content of the composition.

In a preferred embodiment, the magnesium is preferably present in a therapeutic effective amount if the nutritional supplement or composition provides at least 1.2, preferably at least 1.3 times the recommended daily intake (RDI) and preferably not more than 20 times, preferably not more than 10 times the RDI. In a preferred aspect from 1.2 to 5 times, preferably 1.3 to 4.5 times, more preferably 1.4 to 4 times the recommended daily intake is provided. In the EU the RDI based on the ESFA for magnesium is 325 mg per day.

The composition may comprise further minerals including but not limited to sodium, potassium, chloride, calcium, phosphor, iron, zinc, cupper, manganese, fluor, molybdenum, chrome and iodium. Amount thereof comply with general recommendations for nutritional requirements of the FSMP.

### Co-Enzyme Q10

The composition according to the invention preferably comprises therapeutically effective amounts of Co-enzyme Q10. In an embodiment the composition according to the invention comprises DHA, vitamin B2 and D3 in combination with co-enzyme Q10. In a further embodiment, the composition according to the invention may comprise a combination of omega-3 fatty acids DHA and EPA, vitamin B2, vitamin D3, choline, co-enzyme Q10 and one or more of dietary fibres and dietary butyrate.

Co-enzyme Q10 (also known as CoQ10, Q10, vitamin Q10, ubiquinone, and ubidecarenone) is a benzoquinone compound synthesized naturally by the human body. The “Q” and the “10” in the name refer to the quinone chemical group and the 10 isoprenyl chemical subunits, respectively, that are part of this compound's structure. Co-enzyme Q10 as used herein refers to ubiquinone (2,3 dimethoxy-5 methyl-6-decaprenyl benzoquinone) and/or its functional analogues including ubiquinol.

Co-enzyme Q10 is a natural substance soluble in fats that plays a main role as an enzyme cofactor necessary in the transporting chain of mitochondrial electrons, and it is key factor in oxidative phosphorylation and aerobe cell breathing by generating adenosine triphosphate (ATP) (Egil Fosslien; Mitochondrial Medicine—Molecular Pathology of Defective Oxidative Phosphorylation, Annals of Clinical and Laboratory Science, 2000).

CoQ10 was proven to act both as an antioxidant and as an electron carrier due to its lipid solubility and its capacity to exist in both completely reduced forms (ubiquinol) and completely oxidized forms (ubiquinone). CoQ10 accepts electrons derived from both complex I (NADH ubiquinone oxidoreductase) and complex II (succinate ubiquinone reductase), and then transports them to complex III (ubiquinol cytochrome c reductase). Natural sources of CoQ10 and/or analogues that are suitable for this invention include extracts or isolates of fungi, yeasts or bacteria, canola oil or soybean oil.

Preferably, the composition comprises 0.0001 mg to 0.8 mg Co-enzyme Q10, more preferably 0.001 to 0.6 mg Co-enzyme Q10, even more preferably 0.01 to 0.4 mg Co-enzyme Q10 per day. According to one embodiment, Co-enzyme Q10 is preferably provided in an amount of 0.0001 to 0.5 mg per 100 ml, more preferably 0.001 to 0.4 mg per 100 ml, even more preferably 0.001 to 0.3 mg per 100 ml.

According to another embodiment, Co-enzyme Q10 is preferably provided in an amount of 0.0001 to 0.3 mg per 100 kcal of the composition, preferably in amount of 0.001 to 0.3 mg per 100 kcal, even more preferably 0.001 to 0.2 mg per 100 kcal. The above numbers are based on the molar weight of ubiquinone.

### LA/ALA

The lipid fraction may in addition to the omega-3 fatty acid(s) further comprise lipids that are a source of essential fatty acids alpha-linolenic acid (ALA, C-18:3) and linoleic acid (LA, C-18:2). LA and/or ALA may be provided as free fatty acids, in triglyceride form, in diglyceride form, in monoglyceride form, in phospholipid form, or as a mixture of one of more of the above. Preferably the present composition contains at least one, preferably at least two lipid sources selected from the group consisting of soybean oil, rape seed oil (such as colza oil, low erucic acid rape seed oil and canola oil), high oleic sunflower oil, high oleic safflower oil and olive oil. The LA:ALA weight ratio is preferably in the range of 8:1 to 3:1, preferably 7:1 to 4:1, more preferably 6:1 to 5:1.

If present in the composition, the present composition preferably comprises 0.2 to 3.5 g LA per 100 ml, more preferably 0.3 to 3 g LA per 100 ml, more preferably 0.4 to 2.5 g LA per 100 ml. Based on calories, preferably the composition according to the invention may comprise 0.1 to 1.5 g LA per 100 kcal, preferably 0.2 to 1.1 g LA per 100 kcal, more preferably 0.25 to 1 g LA per 100 kcal based on the total energy content of the composition.

If present in the composition, the present composition preferably comprises 0.02 to 0.2 g ALA per 100 ml, more preferably 0.05 to 0.15 g ALA per 100 ml, more preferably 0.06 to 0.1 g ALA per 100 ml. Based on calories, preferably the composition according to the invention may comprise 0.03 to 0.08 g ALA per 100 kcal, preferably 0.04 to 0.07 g ALA per 100 kcal, more preferably 0.05 to 0.065 g ALA per 100 kcal based on the total energy content of the composition.

In an embodiment the composition according to the invention comprises per daily dosage,
(i) 150 to 1200 mg, preferably 200 to 1100 mg, more preferably 250 to 1000 mg DHA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
and one or more of
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and/or
v) 2.5 to 12.5 g, preferably 3 to 12 g, more preferably 3.5 to 11.5 g dietary fibres and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and
v) 2.5 to 12.5 g, preferably 3 to 12 g, more preferably 3.5 to 11.5 g dietary fibres selected from scGOS, IcFOS and low-viscosity pectin and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA and 40 to 350 mg, preferably 50 to 300 mg, more preferably 60 to 250 mg EPA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
and one or more of
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and/or
v) 2.5 to 12.5 g, preferably 3 to 12 g, more preferably 3.5 to 11.5 g dietary fibres and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
and
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine; wherein the composition preferably comprises vₐ) phosphatidylcholine and v_{b}) choline or a salt thereof, and wherein at least 20 mole % choline is provided in the form of phosphatidylcholine, preferably the molar ratio of choline or a salt to PC is in the range from 40:60 to 60:40 and optionally
v) 2.5 to 12.5 g, preferably 3 to 12 g, more preferably 3.5 to 11.5 g dietary fibres and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
in combination with
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and optionally
v) 2.5 to 12.5 g, preferably 3 to 12 g, more preferably 3.5 to 11.5 g dietary fibres selected from scGOS, IcFOS and low-viscosity pectin, preferably, wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA and 40 to 350 mg, preferably 50 to 300 mg, more preferably 60 to 250 mg EPA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
in combination with
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine; wherein choline is selected from free choline, choline salts and/or choline esters, preferably at least phosphatidylcholine; wherein the composition preferably comprises vₐ) phosphatidylcholine and v_{b}) choline or a salt thereof, and wherein at least 20 mole % choline is provided in the form of phosphatidylcholine, preferably the molar ratio of choline or a salt and PC is in the range from 40:60 to 60:40;
and
v) 2.5 to 12.5 g, preferably 3 to 12 g, more preferably 3.5 to 11.5 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA and 40 to 350 mg, preferably 50 to 300 mg, more preferably 60 to 250 mg EPA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
(vi) 0.7 to 9 mg, preferably 0.8 to 8 mg, more preferably 0.9 to 7.5 mg vitamin B6;
(vii) 150 to 900 μg, preferably 175 to 800 μg, more preferably 200 to 750 μg vitamin B9
and one or more of
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and/or
v) 2.5 to 12.5 g, preferably 3 to 12 g, more preferably 3.5 to 11.5 g dietary fibres and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA and 40 to 350 mg, preferably 50 to 300 mg, more preferably 60 to 250 mg EPA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
(vi) 0.7 to 9 mg, preferably 0.8 to 8 mg, more preferably 0.9 to 7.5 mg vitamin B6;
(vii) 150 to 900 μg, preferably 175 to 800 μg, more preferably 200 to 750 μg vitamin B9
and
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine;
and
v) 2.5 to 12.5 g, preferably 3 to 12 g, more preferably 3.5 to 11.5 g dietary fibres selected from scGOS, IcFOS and low-viscosity pectin and/or 0.125 g to 0.65 g dietary butyrate, preferably 0.025 g to 0.65 g, more preferably 0.043 g to 0.65 g dietary butyrate.

In an embodiment the composition according to the invention comprises per daily dosage,
(i) 150 to 1200 mg, preferably 200 to 1100 mg, more preferably 250 to 1000 mg DHA and 40 to 350 mg, preferably 50 to 300 mg, more preferably 60 to 250 mg EPA; with
(ii) 0.8 to 7.6 mg, preferably 1 to 7.4 mg, more preferably in the range of 1.2 to 7.2 mg vitamin B2; with
(iii) 6 to 75 μg, preferably 8 to 70 μg, more preferably 10 to 65 μg vitamin D3;
(vi) 0.7 to 9 mg, preferably 0.8 to 8 mg, more preferably 0.9 to 7.5 mg vitamin B6;
(vii) 150 to 900 μg, preferably 175 to 800 μg, more preferably 200 to 750 μg vitamin B9;
and
(iv) 200 to 1400 mg, preferably 300 to 1200 mg, more preferably 350 to 1100 mg choline based on molar weight of choline, wherein choline is selected from free choline, choline salts and/or choline esters, preferably at least phosphatidylcholine; wherein choline is selected from free choline, choline salts and/or choline esters, preferably at least phosphatidylcholine; wherein the composition preferably comprises vₐ) phosphatidylcholine and v_{b}) choline or a salt thereof, and wherein at least 20 mole % choline is provided in the form of phosphatidylcholine, preferably the molar ratio of choline or a salt to PC is in the range from 40:60 to 60:40;
and
v) 2.5 to 12.5 g, preferably 3 to 12 g, more preferably 3.5 to 11.5 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.125 g to 0.65 g dietary butyrate, preferably 0.025 g to 0.65 g, more preferably 0.043 g to 0.65 g dietary butyrate.

In a further preferred embodiment the composition as embodied above comprising per daily dosage a combination of (i) (ii) and (iii), at least one of (vi) and (vii) and optionally (iv) and/or (v) further comprises one or more of
(viii) 350 to 2700 μg Retinol Equivalents, preferably 650 to 900 μg Retinol Equivalents, more preferably 375 to 2600 μg Retinol Equivalents of vitamin A;
(ix) 0.7 to 5 mg, preferably 0.8 to 4.9 mg, more preferably 0.9 to 4.8 mg vitamin B1;
(x) 2 to 20 μg, preferably 2.5 to 19 μg, more preferably 3 to 18 μg vitamin B12;
(xi) 75 to 700 mg, preferably 85 to 600 mg, more preferably 95 to 550 mg vitamin C;
(xii) 8 to 75 mg, preferably 10 to 70 mg, more preferably 12.5 to 70 mg alpha-TE;
(xiii) 30 to 275 μg, preferably 35 to 250 μg, more preferably 38 to 225 μg selenium; and,
(xiv) 125 to 850 mg, preferably 150 to 800 mg, more preferably 170 to 750 mg magnesium
(xv) 0.0001 mg to 0.8 mg Co-enzyme Q10, preferably 0.001 to 0.6 mg Co-enzyme Q10, even more preferably 0.01 to 0.4 mg Co-enzyme Q10.

In a further preferred embodiment the composition comprising per daily dosage a combination of (i) (ii), (iii), (iv) and (v) at least one of (vi) and (vii) and one or more of
(viii) 350 to 2700 μg Retinol Equivalents, preferably 650 to 900 μg Retinol Equivalents, more preferably 375 to 2600 μg Retinol Equivalents of vitamin A;
(ix) 0.7 to 5 mg, preferably 0.8 to 4.9 mg, more preferably 0.9 to 4.8 mg vitamin B1;
(x) 2 to 20 μg, preferably 2.5 to 19 μg, more preferably 3 to 18 μg vitamin B12;
(xi) 75 to 700 mg, preferably 85 to 600 mg, more preferably 95 to 550 mg vitamin C;
(xii) 8 to 75 mg, preferably 10 to 70 mg, more preferably 12.5 to 70 mg alpha-TE;
(xiii) 30 to 275 μg, preferably 35 to 250 μg, more preferably 38 to 225 μg selenium; and,
(xiv) 125 to 850 mg, preferably 150 to 800 mg, more preferably 170 to 750 mg magnesium
(xv) 0.0001 mg to 0.8 mg Co-enzyme Q10, preferably 0.001 to 0.6 mg Co-enzyme Q10, even more preferably 0.01 to 0.4 mg Co-enzyme Q10

In a further preferred embodiment the composition comprises per daily dosage a combination of (i) (ii) and (iii), at least one of (vi) and (vii) and optionally (iv) and/or (v), one or more selected of (vii)-(xv) further comprises at least one of
(xvi) 3 to 45 mg, preferably 3.5 to 42 mg, more preferably 3.8 to 40 mg vitamin B3 (niacin equivalents);
(xvii) 0.8 to 15 mg, preferably 1 to 14 mg, more preferably 1.2 to 13 mg vitamin B5;
(xviii) 8 to 95 μg, preferably in the range of 9 to 90 μg, more preferably in the range of 9.5 to 85 μg vitamin B7.

Provided herein is also a liquid nutritional composition comprising per 100 ml:
(i) 30 to 240 mg, preferably 35 to 220 mg, more preferably 40 to 200 mg DHA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and one or more of
(iv) 35 to 170 mg, preferably 40 to 160 mg, more preferably 45 to 150 mg choline, wherein choline is selected from free choline, choline salts and/or choline esters, preferably at least phosphatidylcholine;
and/or
v) 1.5 to 4.5 g, preferably 2 to 4 g, more preferably 2.5 to 3.5 g dietary fibres and/or 0.01 to 0.175 g dietary butyrate, preferably 0.02 to 0.175 g, more preferably 0.035 to 0.175 g dietary butyrate.

Provided herein is also a liquid nutritional composition comprising per 100 ml:
(i) 30 to 240 mg, preferably 35 to 220 mg, more preferably 40 to 200 mg DHA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and
(iv) 35 to 170 mg, preferably 40 to 160 mg, more preferably 45 to 150 mg choline, wherein choline is selected from free choline, choline salts and/or choline esters, preferably at least phosphatidylcholine;
and
v) 0.5 to 4.0 g, preferably 1.0 to 3.5 g, more preferably 1.5 to 3.0 g dietary fibres and/or 0.01 to 0.175 g dietary butyrate, preferably 0.02 to 0.175 g, more preferably 0.035 to 0.175 g dietary butyrate.

Provided herein is also a liquid nutritional composition comprising per 100 ml:
(i) 30 to 240 mg, preferably 35 to 220 mg, more preferably 40 to 200 mg DHA and 5 to 60 mg, preferably 7 to 55 mg, more preferably 9 to 50 mg EPA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and one or more of
(iv) 35 to 170 mg, preferably 40 to 160 mg, more preferably 45 to 150 mg choline, wherein choline is selected from free choline, choline salts and/or choline esters, preferably at least phosphatidylcholine;
and/or
v) 0.5 to 4.0 g, preferably 1.0 to 3.5 g, more preferably 1.5 to 3.0 g dietary fibres and/or 0.01 to 0.175 g dietary butyrate, preferably 0.02 to 0.175 g, more preferably 0.035 to 0.175 g dietary butyrate

Provided herein is also a liquid nutritional composition comprising per 100 ml:
(i) 30 to 240 mg, preferably 35 to 220 mg, more preferably 40 to 200 mg DHA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and
(iv) 35 to 170 mg, preferably 40 to 160 mg, more preferably 45 to 150 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt to PC is in the range from 40:60 to 60:40;
and optionally
v) 1.5 to 4.5 g, preferably 2 to 4 g, more preferably 2.5 to 3.5 g dietary fibres and/or 0.01 to 0.175 g dietary butyrate, preferably 0.02 to 0.175 g, more preferably 0.035 to 0.175 g dietary butyrate.

Provided herein is also a liquid nutritional composition comprising per 100 ml:
(i) 30 to 240 mg, preferably 35 to 220 mg, more preferably 40 to 200 mg DHA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
in combination with
(iv) 35 to 170 mg, preferably 40 to 160 mg, more preferably 45 to 150 mg choline, wherein choline is selected from free choline, choline salts and/or choline esters, preferably at least phosphatidylcholine; and/or
v) 0.5 to 4.0 g, preferably 1.0 to 3.5 g, more preferably 1.5 to 3.0 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.01 to 0.175 g dietary butyrate, preferably 0.02 to 0.175 g, more preferably 0.035 to 0.175 g dietary butyrate.

Provided herein is also a liquid nutritional composition comprising per 100 ml:
(i) 30 to 240 mg, preferably 35 to 220 mg, more preferably 40 to 200 mg DHA and 5 to 60 mg, preferably 7 to 55 mg, more preferably 9 to 50 mg EPA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and
(iv) 35 to 170 mg, preferably 40 to 160 mg, more preferably 45 to 150 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt to PC is in the range from 40:60 to 60:40;
and
v) 0.5 to 4.0 g, preferably 1.0 to 3.5 g, more preferably 1.5 to 3.0 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.01 to 0.175 g dietary butyrate, preferably 0.02 to 0.175 g, more preferably 0.035 to 0.175 g dietary butyrate

Provided herein is also a liquid nutritional composition comprising per 100 ml:
(i) 30 to 240 mg, preferably 35 to 220 mg, more preferably 40 to 200 mg DHA and 5 to 60 mg, preferably 7 to 55 mg, more preferably 9 to 50 mg EPA; with
(ii) 0.1 to 1.4 mg, preferably 0.2 to 1.2 mg, more preferably 0.3 to 1 mg vitamin B2; with
(iii) 1.5 to 10 μg, preferably 2 to 9 μg, more preferably 2.5 to 8 μg vitamin D3;
and one or more of
(iv) 35 to 170 mg, preferably 40 to 160 mg, more preferably 45 to 150 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt and PC is in the range from 40:60 to 60:40;
and/or
v) 0.5 to 4.0 g, preferably 1.0 to 3.5 g, more preferably 1.5 to 3.0 g dietary fibres selected from scGOS, IcFOS and low-viscosity pectin, preferably wherein the dietary fibres comprises a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.01 to 0.175 g dietary butyrate, preferably 0.02 to 0.175 g, more preferably 0.035 to 0.175 g dietary butyrate
and at least one of (vi) and (vii);
(vi) 0.2 to 1 mg, preferably 0.25 to 0.8 mg, more preferably 0.3 to 0.7 mg vitamin B6;
(vii) 20 to 225 μg, preferably 25 to 200 μg, more preferably 30 to 175 μg vitamin B9.

In a further preferred embodiment the liquid nutritional composition comprising per 100 ml a combination of (i) and (ii), (iii), at least one of (vi) and (vii) and optionally (iv) and/or to (v), further comprises one or more of
(viii) 50 to 400 μg, preferably 75 to 350 μg, more preferably 100 to 300 μg Retinol Equivalents of vitamin A;
(ix) 0.1 to 0.9 mg, preferably 0.15 to 0.8 mg, more preferably 0.2 to 0.75 mg vitamin B1;
(x) 0.5 to 3 μg, preferably 0.6 to 2.6 μg, more preferably 0.7 to 2.4 μg vitamin B12;
(xi) 10 to 100 mg, preferably 15 to 90 mg, more preferably 20 to 80 mg vitamin C;
(xii) 2 to 14 mg, preferably 2.5 to 12 mg, more preferably 2.75 to 10 mg alpha-TE;
(xiii) 8 to 45 μg, preferably 10 to 40 μg, more preferably 11 to 35 μg selenium;
(xiv) 20 to 160 mg, preferably 25 to 150 mg, more preferably 30 to 140 mg magnesium; and
(xv) 0.0001 to 0.5 mg, preferably 0.001 to 0.4 mg, more preferably 0.001 to 0.3 mg Co-enzyme Q10.

In a further preferred embodiment the liquid nutritional composition may further comprise per 100 ml one of more of
(xvi) 1.2 to 6 mg, preferably 1.4 to 5.5 mg, more preferably 1.6 to 5 mg vitamin B3 (niacin equivalents);
(xvii) 0.3 to 2.2 mg. preferably 0.4 to 2 mg, more preferably 0.5 to 1.8 mg vitamin B5;
(xviii) 2 to 14 mg, preferably 3 to 12 mg, more preferably 3.5 to 11 mg vitamin B7.

Provided herein is further a liquid nutritional composition comprising per 100 kcal:
(i) 20 to 100 mg, preferably 25 to 90 mg, more preferably 30 to 80 mg DHA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
and one or more of
(iv) 100 to 1000 mg, preferably 200 to 800 mg, more preferably 300 to 500 mg choline, wherein choline is selected from free choline, choline salts and/or choline esters, preferably at least phosphatidylcholine;
and/or
v) 0.75 to 2.5 g, preferably 1.0 to 2.0 g, more preferably 1.1 to 1.5 g dietary fibres and/or 0.015 to 0.25 g dietary butyrate, preferably 0.03 to 0.25 g, more preferably 0.05 to 0.25 g dietary butyrate.

Provided herein is further a liquid nutritional composition comprising per 100 kcal:
(i) 20 to 100 mg, preferably 25 to 90 mg, more preferably 30 to 80 mg DHA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
and
(iv) 100 to 1000 mg, preferably 200 to 800 mg, more preferably 300 to 500 mg choline, wherein choline is selected from free choline, choline salts and/or choline esters, preferably at least phosphatidylcholine;
and
v) 0.75 to 2.5 g, preferably 1.0 to 2.0 g, more preferably 1.1 to 1.5 g dietary fibres and/or 0.015 to 0.25 g dietary butyrate, preferably 0.03 to 0.25 g, more preferably 0.05 to 0.25 g dietary butyrate.

Provided herein is further a liquid nutritional composition comprising per 100 kcal:
(i) 20 to 100 mg, preferably 25 to 90 mg, more preferably 30 to 80 mg DHA and 5 to 30 mg, preferably 6 to 25 mg, more preferably 8 to 20 mg EPA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
and one or more of
(iv) 100 to 1000 mg, preferably 200 to 800 mg, more preferably 300 to 500 mg choline, wherein choline is selected from free choline, choline salts and/or choline esters, preferably at least phosphatidylcholine
and/or
v) 0.75 to 2.5 g, preferably 1.0 to 2.0 g, more preferably 1.1 to 1.5 g dietary fibres and/or 0.015 to 0.25 g dietary butyrate, preferably 0.03 to 0.25 g, more preferably 0.05 to 0.25 g dietary butyrate.

Provided herein is further a liquid nutritional composition comprising per 100 kcal:
(i) 20 to 100 mg, preferably 25 to 90 mg, more preferably 30 to 80 mg DHA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
and
(iv) 100 to 1000 mg, preferably 200 to 800 mg, more preferably 300 to 500 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt and PC is in the range from 40:60 to 60:40;
and optionally
v) 0.75 to 2.5 g, preferably 1.0 to 2.0 g, more preferably 1.1 to 1.5 g dietary fibres and/or 0.015 to 0.25 g dietary butyrate, preferably 0.03 to 0.25 g, more preferably 0.05 to 0.25 g dietary butyrate.

Provided herein is further a liquid nutritional composition comprising per 100 kcal:
(i) 20 to 100 mg, preferably 25 to 90 mg, more preferably 30 to 80 mg DHA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
in combination with
(iv) 100 to 1000 mg, preferably 200 to 800 mg, more preferably 300 to 500 mg choline, wherein choline is selected from free choline, choline salts and/or choline esters, preferably at least phosphatidylcholine;
and/or
v) 0.75 to 2.5 g, preferably 1.0 to 2.0 g, more preferably 1.1 to 1.5 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.015 to 0.25 g dietary butyrate, preferably 0.03 to 0.25 g, more preferably 0.05 to 0.25 g dietary butyrate.

Provided herein is further a liquid nutritional composition comprising per 100 kcal:
(i) 20 to 100 mg, preferably 25 to 90 mg, more preferably 30 to 80 mg DHA 5 to 30 mg, preferably 6 to 25 mg, more preferably 8 to 20 mg EPA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
in combination with
(iv) 100 to 1000 mg, preferably 200 to 800 mg, more preferably 300 to 500 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt to PC is in the range from 40:60 to 60:40;
and
v) 0.75 to 2.5 g, preferably 1.0 to 2.0 g, more preferably 1.1 to 1.5 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.015 to 0.25 g dietary butyrate, preferably 0.03 to 0.25 g, more preferably 0.05 to 0.25 g dietary butyrate

Provided herein is further a liquid nutritional composition comprising per 100 kcal:
(i) 20 to 100 mg, preferably 25 to 90 mg, more preferably 30 to 80 mg DHA 5 to 30 mg, preferably 6 to 25 mg, more preferably 8 to 20 mg EPA; with
(ii) 0.4 to 0.6 mg, preferably 0.15 to 0.5 mg, more preferably 0.2 to 0.4 mg vitamin B2; with
(iii) 1.3 to 4.5 μg, preferably 1.5 to 4 μg, more preferably 2 to 3.5 μg vitamin D3;
in combination with at least one of (vi) and (vii);
(vi) 0.1 to 0.5 mg, preferably 0.2 to 0.4 mg, more preferably 0.25 to 0.3 mg vitamin B6;
(vii 16 to 80 μg, preferably 18 to 75 μg, more preferably 20 to 70 μg vitamin B9;
and one or more of
(iv) 100 to 1000 mg, preferably 200 to 800 mg, more preferably 300 to 500 mg choline, wherein choline is selected from free choline, choline salts and/or choline esters, preferably at least phosphatidylcholine; and wherein the composition preferably comprises vₐ) phosphatidylcholine and v_{b}) choline or a salt thereof and wherein at least 20 mole % choline is provided in the form of phosphatidylcholine, preferably the molar ratio of choline or a salt and PC is in the range from 40:60 to 60:40;
and/or
v) 0.75 to 2.5 g, preferably 1.0 to 2.0 g, more preferably 1.1 to 1.5 g dietary fibres selected from scGOS, IcFOS and low-viscosity pectin, preferably wherein the dietary fibres comprise a mixture of scGOS, IcFOS and low viscosity pectin and wherein the ratio scGOS:IcFOS:low-viscosity pectin is in the range of 5:1:1 to 12:1:3, more preferably in the range of 7:1:1.5 to 11:1:2.5, most preferably, in the range of 8:1:1.5 to 10:1:2.5 and/or 0.015 to 0.25 g dietary butyrate, preferably 0.03 to 0.25 g, more preferably 0.05 to 0.25 g dietary butyrate.

In a further preferred embodiment the liquid nutritional composition comprises per 100 kcal a combination of (i) (ii) and (iii), at least one of (vi) and (vii) and optionally (iv) and/or to (v), further comprises one or more of
(viii) 60 to 150 μg, preferably 70 to 140 μg, more preferably 80 to 130 μg Retinol Equivalents of vitamin A;
(ix) 0.05 to 0.4 mg, preferably 0.1 to 0.35 mg, more preferably 0.15 to 0.30 mg vitamin B1;
(x) 0.4 to 1.4 μg, preferably 0.5 to 1.2 μg, more preferably 0.6 to 1 μg vitamin B12;
(xi) 12 to 40 mg, preferably 14 to 35 mg, more preferably 16 to 32 mg vitamin C;
(xii) 1 to 5.5 mg, preferably 1.5 to 5 mg, more preferably 2 to 4.5 mg alpha-TE;
(xiii) 4 to 18 μg, preferably 6 to 16 μg, more preferably 8 to 14 μg selenium; and,
(xiv) 10 to 70 mg, preferably 15 to 65 mg, more preferably 20 to 60 mg magnesium
(xv) 0.0001 to 0.3 mg, preferably 0.001 to 0.3 mg, more preferably 0.001 to 0.2 mg Co-enzyme Q10.

In a further preferred embodiment the liquid nutritional composition may further comprise per 100 kcal one of more of
(xv) 1 to 2.5 mg, preferably 1.1 to 2.2 mg, more preferably 1.2 to 2 mg vitamin B3 (niacin equivalents);
(xvi) 0.2 to 1 mg. preferably 0.3 to 0.9 mg, more preferably 0.4 to 0.8 mg vitamin B5;
(xvii) 1.5 to 6 mg, preferably 2 to 5.5 mg, more preferably 2.5 to 5 mg vitamin B7;

In a particular embodiment there is provided a composition comprising
(i) DHA and EPA;
(ii) vitamin D3;
(iii) vitamin B2;
(iv) choline, choline salts and/or choline ester, preferably at least phosphatidylcholine;
(v) dietary fibres and/or dietary butyrate;
(vi) vitamin B6;
(vii) vitamin B9;
and at least one or more of the following ingredients
(viii) Retinol Equivalents of vitamin A;
(ix) vitamin B1;
(x) vitamin B12;
(xi) vitamin C;
(xii) alpha-TE;
(xiii) selenium; and,
(xiv) magnesium
(xv) co-enzyme Q10.

In the most preferred embodiment, the nutritional composition comprises a combination of
i) DHA; preferably in the range of 480-680 mg DHA, more preferably 510-650 mg, most preferably 550-610 mg based on 100 g dry weight of the composition;
ii) vitamin B2; preferably in the range of 1.23-1.53 mg, more preferably 1.27-1.49 mg, most preferably 1.31-1.44 mg based on 100 g dry weight of the composition;
iii) vitamin D3; preferably in the range of 38-54 μg, more preferably 40-52 μg, most preferably 42-50 μg based on 100 g dry weight of the composition;
iv) choline; preferably 200-370 mg choline, more preferably 230-340 mg, most preferably 250-310 mg choline based on 100 g dry weight of the composition
v) dietary fibres; preferably in the range of 9.0-11.3 g, more preferably 9.2-11 g, most preferably 9.8-10.8 g based on 100 g dry weight of the composition.

The nutritional composition preferably further comprises EPA, preferably in the range of 100-170 mg, more preferably 110-160 mg, most preferably 120-150 mg EPA based on 100 g dry weight of the nutritional composition. Preferably, choline is provided in the form of free choline, a choline salt and/or choline ester, wherein the choline ester is preferably phosphatidylcholine, more preferably choline is provided in the form of a choline salt and phosphatidylcholine. Preferably, at least 1-5 wt % of choline in the nutritional composition is provided in the form of phosphatidylcholine. In a preferred embodiment, choline as provided in the form of phosphatidylcholine is preferably in the range of 5-15 mg, more preferably 6-12 mg, most preferably 7-10 mg choline based on 100 g dry weight of the composition Preferably phosphatidylcholine is comprised in phospholipids and the nutritional composition preferably comprises 200-290 mg phospholipids, more preferably 210-280 mg, most preferably 220-265 mg phospholipids based on 100 g dry weight of the composition. Preferably, the composition is a powder which is reconstitutable with a liquid, preferably with water, to obtain a liquid composition.

In the most preferred embodiment, the nutritional composition comprises based on 100 ml:
i) 30 to 240 mg DHA, preferably 35 to 220 mg, more preferably 40 to 200 mg DHA;
ii) 0.15-1.4 mg vitamin B2, preferably 0.2-1.2 mg, more preferably 0.3-1 mg vitamin B2
iii) 6-20 μg vitamin D3, preferably 8-18 μg, more preferably 10-15 μg vitamin D3;
iv) 30 to 180 mg choline, preferably 35 to 170 mg, more preferably 40 to 160 mg choline;
v) 0.5 to 4.0 g dietary fibers, preferably 1.0 to 3.5 g, most preferably 1.5 to 3.0 g dietary fibres.

The nutritional composition preferably further comprises EPA, preferably in the range of 5 to 60 mg, more preferably 7 to 55 mg, most preferably 9 to 50 mg EPA per 100 ml of the composition. Preferably, choline is provided in the form of free choline, a choline salt and/or choline ester, wherein said choline ester is preferably phosphatidylcholine, more preferably choline is provided in the form of a choline salt and phosphatidylcholine. Choline provided in the form of phosphatidylcholine is preferably in the range of 0.5-8 mg, more preferably 1-7 mg, most preferably 1.5-5 mg choline per 100 ml of the composition. Also, phosphatidylcholine is part of phospholipids and the nutritional composition preferably comprises 20-200 mg phospholipids, more preferably 40-150 mg, most preferably 50-100 mg phospholipids per 100 ml of the composition.

In the most preferred embodiment, the nutritional composition comprises per 100 kcal based on the total energy content of the nutritional composition:
i) 30 to 240 mg DHA, preferably 35 to 220 mg, more preferably 40 to 200 mg DHA;
ii) 0.1-1.4 mg vitamin B2, preferably 0.2-1.2 mg, more preferably 0.15-0.5 mg vitamin B2
iii) 5-30 μg vitamin D3, preferably 8-20 μg, more preferably 10-17 μg vitamin D3;
iv 25 to 110 mg choline, preferably 30 to 105 mg, more preferably 33 to 100 mg choline;
v) 0.5 to 4.0 g dietary fibers, preferably 1.0 to 3.5 g, most preferably 1.5 to 3.0 g dietary fibres.

The nutritional composition preferably further comprises EPA, preferably in the range of comprises 5 to 60 mg, more preferably 7 to 55 mg, most preferably 9 to 50 mg EPA per 100 kcal based on total energy content of the composition. Preferably, choline is provided in the form of free choline, a choline salt and/or choline ester, wherein the choline ester is preferably phosphatidylcholine, more preferably choline is provided in the form of a choline salt and phosphatidylcholine. Choline derived from phosphatidylcholine is preferably in the range of 0.5-8 mg, more preferably 1-7 mg, most preferably 1.5-5 mg choline per 100 kcal based on total energy of the composition. Also, phosphatidylcholine is part of phospholipids and the nutritional composition preferably comprises 20-200 mg phospholipids, more preferably 40-150 mg, most preferably 50-100 mg phospholipids per 100 kcal based on total energy of the composition.

In the most preferred embodiment, the nutritional composition comprises per daily dosage:
i) 100 to 1250 mg DHA, preferably 150 to 1200 mg, more preferably 200 to 1100 mg, most preferably 250 to 1000 mg;
ii) 0.2 to 10 mg vitamin B2, preferably 0.4 to 8 mg, more preferably 0.8 to 7.6 mg vitamin B2;
iii) 5 to 80 μg vitamin D3, preferably 6 to 75 μg, more preferably 8 to 70 μg vitamin D3;
iv) 70 to 1300 mg choline, preferably 80 to 1200 mg, more preferably 100 to 1100 mg choline, wherein choline comprises at least phosphatidylcholine and a choline salt;
v) 2.5 to 12.5 g dietary fibres, preferably 3 to 12 g, more preferably 3.5 to 11.5 g dietary fibres.

The nutritional composition preferably further comprises 40 to 350 mg, preferably 50 to 300 mg, more preferably 60 to 250 mg EPA per day. In a preferred embodiment, choline as derived from phosphatidylcholine is preferably in the range of 1 to 15 mg, preferably 2 to 12 mg, more preferably 3 to 10 mg choline per day. Also, phosphatidylcholine is part of phospholipids and the nutritional composition preferably comprises 50 to 350 mg phospholipids, more preferably 75 to 300 mg, even more preferably 90 to 250 mg phospholipids per day.

### EXAMPLES

For a more complete understanding of the present disclosure, reference is now made to the following examples taken in conjunction with the accompanying drawings.

### Example 1—Synergy Between DHA and EPA, B2 and D3 on Neurite Outgrowth in PC12 Cells

### PC12 Neuronal Cell Cultures

In order to assess the effects of nutritional components on axonal outgrowth and dendritic arborization underlying functional brain connectivity, the effects of nutrient supplementation on neurite outgrowth parameters in PC12 pheochromocytoma cells was investigated. PC12 neuronal cell model is one of the most widely used cell models for the study of neuronal differentiation.

PC12 pheochromocytoma cells were grown in DMEM (Gibco), supplemented with 10% fetal bovine serum (FBS), penicillin (100 units/ml), and streptomycin (100 μg/ml) (Gibco), under a humidified atmosphere with 95% air and 5% CO2 at 37° C. Cells were seeded onto a collagen (0.01 mg/ml) coated 96-well plates at a density of 5000 cells/well and after 24 hours supplemented with medium containing 20 ng/ml nerve growth factor (NGF) with or without the components as defined in the present and in the following examples. Cell cultures were incubated at 37° C. in 5% CO2 and medium was replaced every 2-3 days.

### Nutrient Supplementation

The effects of nutrient supplementation of neuronal PC12 cultures were assessed by comparing different concentrations of DHA (3.3 μM and 6.7 μM), EPA (10 μM), vitamin B2 (0.5 μM and 1 μM) and vitamin D3 (0.25 μM) to the vehicle control (0 μM). Nutrients were supplemented either alone or in combination with each other.

DHA and EPA were resuspended in absolute ethanol and diluted 5 times in fetal bovine serum. Other compounds were dissolved in DMEM. All compounds were purchased from Sigma (Zwijndrecht, The Netherlands). Supplementation of cells was performed in 6-fold in 96 wells plates. Neurite outgrowth of these conditions was compared to non-supplemented cells.

### Neurite Outgrowth: Immunocytochemistry and Imaging

After 48 hours of nutrient supplementation, cell bodies and neurites were stained using Calcein-AM stain (0.2 μg/ml) (Invitrogen, batch #C3100MP) and the nuclei were counterstained using Hoechst stain (1.2 μg/ml) (Thermo Scientific, batch #62249) in 100 μl culture medium per well and incubated for 20 min at 37° C. Subsequently, FITC and DAPI images (9 images per well, 6 wells per condition) were taken using an ImageXpress Micro Confocal high content imaging system (Molecular Devices). Neurite length was quantified using the neurite outgrowth module in MetaXpress. Image analysis was carried out using the neurite outgrowth analysis module in MetaXpress (Molecular Devices). The measurements taken from each well included the number of nuclei and the mean neurite outgrowth (μm) per cell. The data was normalized to and expressed as a relative percentage of the plate-averaged value of the vehicle-treated control wells.

### Results

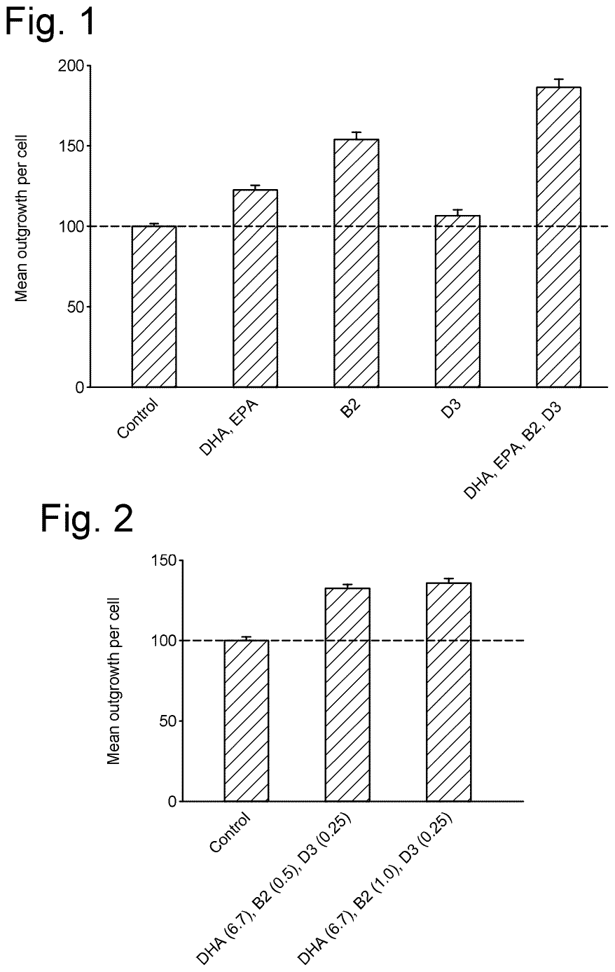

Image analysis of the neurite outgrowth showed that supplementation of omega-3 fatty acids DHA and EPA, vitamin B2 and D3 alone increased neurite outgrowth as compared to control in an in vitro PC12 neuronal cell model. Neurite outgrowth is expressed as percentage relative to control (100%). Furthermore, when taken together, the neurite outgrowth improved in a synergistical way: the mean percentage of neurite outgrowth was higher than expected based on the additional effect of each component (mean calculated or expected neurite outgrowth was 183.1, the observed mean neurite outgrowth was 186.4). These findings are depicted in FIG. 1.

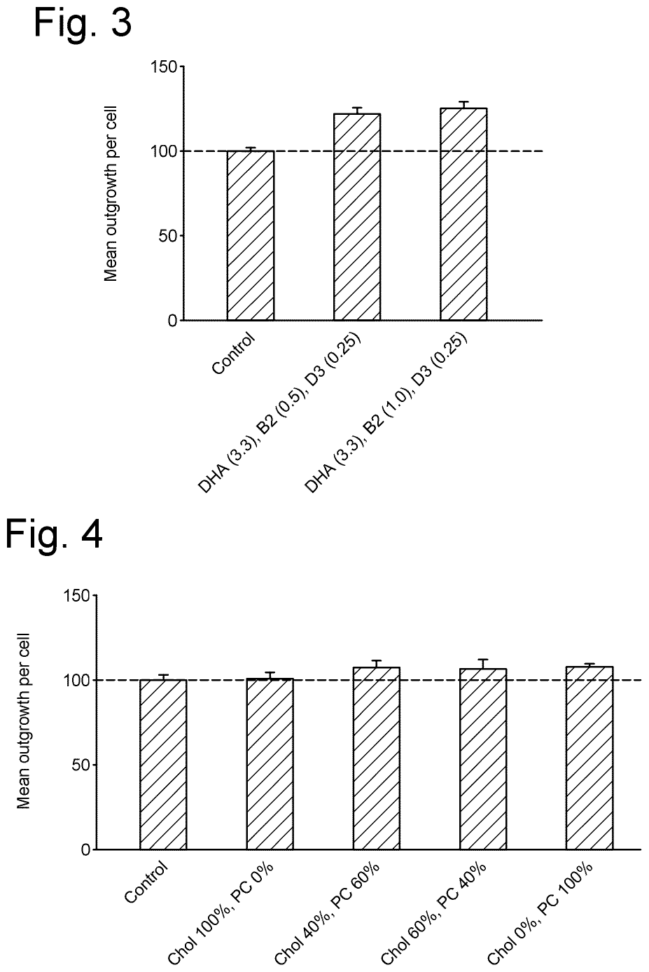

FIG. 2 performed on a separate 96-well plate, in which supplementation was with DHA (6.7 μM) as sole omega-3 fatty acid and in which the concentration of vitamin B2 varied between 0.5 μM and 1 μM, showed a similar improved trend on neurite outgrowth as compared to the control. Similar effects are shown in FIG. 3 in which the concentration of DHA was 3.3 μM and of vitamin B2 was varied between 0.5 μM and 1 μM. Together these findings show that the effect on neurite outgrowth is present over a broad concentration range of DHA and vitamin B2.

It should be noted that in-between plate comparison cannot be done as the neurite outgrowth is a relative measure compared to the vehicle-supplemented control and thus only intra-plate variation should be taken into account.

### Example 2—Ratio Choline-Chloride:PC Impacts Neurite Outgrowth in PC12 Cells

Reference is made to example 1 for PC12 neuronal cell cultures and measurement of neurite outgrowth with immunohistochemistry and imaging.

### Nutrient Supplementation

The effects of nutrient supplementation with different sources of choline by supplementation with either choline-chloride (chol-Cl) or phosphatidylcholine (PC) in neuronal cultures was assessed by comparing different molar ratios of chol-Cl and PC to the vehicle control (0 μM). Nutrients were supplemented alone (100 mole % chol-Cl or 100 mole % PC) and in a molar ratio of 60:40 and 40:60 of chol-Cl:PC. Choline supplementation of cells was performed in 6-fold in 96 wells plates at a total concentration of 20 μM choline, derived from chol-Cl, from PC, or from both, wherein the total amount of choline was 20 μM. These conditions were compared to vehicle-supplemented control cells.

### Results

Image analysis of the neurite outgrowth showed that supplementation with 100% PC increases neurite outgrowth more than supplementation with 100 mole % chol-Cl compared to control. In addition, a molar ratio of chol-Cl:PC between 60:40 and 40:60 showed similar improved neurite outgrowth compared to control and compared to supplementation of 100 mole % chol-Cl. These findings are depicted in FIG. 4.

The neurite outgrowth results indicate that both choline chloride and PC enhance neurite outgrowth. Choline provided in the form of PC in combination with chol-Cl slightly enhanced the effect on neurite outgrowth.

### Example 3—Synergy Between DHA (and EPA), B2 and D3 and Chol-Cl and/or PC on Neurite Outgrowth in PC12 Cells

Reference is made to example 1 for PC12 neuronal cell cultures and measurement of neurite outgrowth with immunohistochemistry and imaging.

### Nutrient Supplementation

The effects of nutrient supplementation of chol-Cl, PC and combinations of DHA, B2 and D3 on neurite outgrowth of PC12 cells were assessed by comparison to the vehicle control. PC12 cells were supplemented with the following nutrients:
(1) 100% chol-Cl (20 μM choline),
(2) 100% PC (20 μM choline),
(3) a combination of DHA (3.3 μM), B2 (0.5 μM) and D3 (0.25 μM),
(4) a combination of 100% chol-Cl (20 μM), DHA (3.3 μM), B2 (0.5 μM) and D3 (0.25 μM),
(5) a combination of 80 mole % chol-Cl+20 mole % PC (total of 20 μM), DHA (3.3 μM), B2 (0.5 μM) and D3 (0.25 μM)
(6) a combination of 100% PC (20 μM), DHA (3.3 μM), B2 (0.5 μM) and D3 (0.25 μM).

In a separate experiment PC12 cells were supplemented with PC (20 μM, resulting in 2.8 μM choline) alone, the combination of DHA (5 μM) and EPA (1 μM), B2 (0.5 μM) and D3 (0.25 μM) and the combination of PC (20 μM), DHA (5 μM) and EPA (1 μM), B2 (0.5 μM) and D3 (0.25 μM).

In all relevant conditions, the amount of choline whether derived from chol-Cl or PC was kept constant at a concentration of 20 μM. Supplementation of cells was performed in 6-fold in 96 wells plates. These conditions were compared to non-supplemented cells.

### Results

Again, image analysis of the neurite outgrowth showed that supplementation with 100 mole % PC increased neurite outgrowth more than supplementation with 100 mole % chol-Cl compared to control. In addition, the combination of DHA, B2 and D3 beneficially further increased neurite outgrowth.

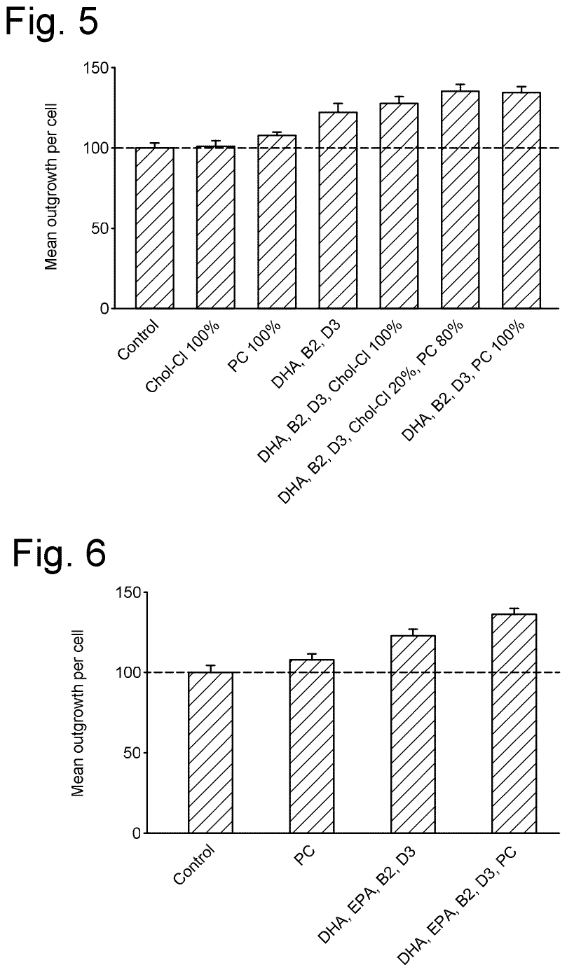

Unexpectedly, the combination of 100 mole % chol-Cl with DHA, B2 and D3 synergistically increased neurite outgrowth more than predicted based on the additional effect of chol-Cl alone and the combination of DHA, B2 and D3 (mean calculated or expected neurite outgrowth was 122.9, the observed mean neurite outgrowth was 127.7). The same effect was observed for the combination of 100 mole % PC with DHA, B2 and D3 (mean calculated or expected neurite outgrowth was 129.8, the observed mean neurite outgrowth was 134.3). The synergistic effect remained present at a molar ratio of 20:80 of chol-Cl to PC combined with DHA, B2 and D3 (observed mean neurite outgrowth was 135.2). These findings are depicted in FIG. 5.

Furthermore, FIG. 6 shows the effect of PC combined with DHA and EPA, B2 and D3 on neurite outgrowth in PC12 cells. Again, a synergistic effect was observed when comparing the effect of PC alone to the combination of DHA, EPA, B2 and D3 (mean calculated or expected neurite outgrowth was 130.7, the observed mean neurite outgrowth was 136.3).

These results indicate that the improved neurite outgrowth is present when combined with chol-Cl or with PC and the effect is present over a range of concentrations of DHA and also in the presence of EPA as omega-3 FA.

### Example 4—BA Promotes Neurite Outgrowth in PC12 Cells in a Dose-Dependent Proportional Effect

Reference is made to example 1 for PC12 neuronal cell cultures and measurement of neurite outgrowth with immunohistochemistry and imaging.

### Nutrient Supplementation

The effects of nutrient supplementation of BA in neuronal cell culture was assessed by comparing different concentrations of butyric acid (BA) (12.5 μM, 25 μM, 50 μM, 100 μM and 200 μM) to the vehicle control (0 μM). Supplementation of cells was performed in 6-fold in 96 wells plates. These conditions were compared to non-supplemented cells.

### Results

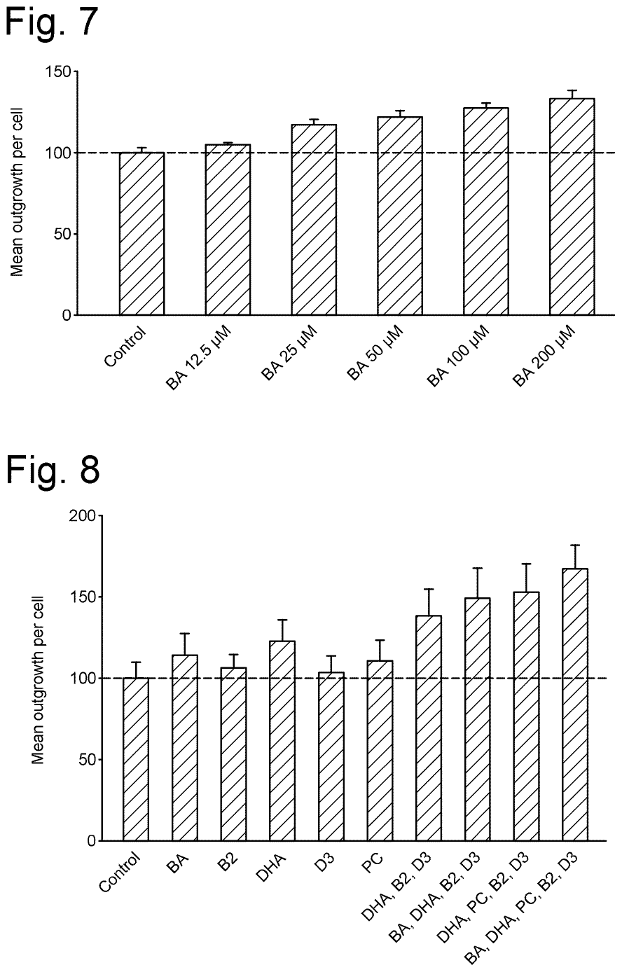

Image analysis of the neurite outgrowth showed that supplementation with BA increased neurite outgrowth in a dose-dependent manner. The findings are depicted in FIG. 7. A dose-response effect was observed and at a concentration of 200 μM BA the strongest effect on neurite outgrowth was observed.

### Example 5—Synergy Between DHA, B2, D3, PC and BA on Neurite Outgrowth in PC12 Cells

Reference is made to example 1 for PC12 neuronal cell cultures and measuring neurite outgrowth with immunohistochemistry and imaging.

### Nutrient Supplementation

The effects of nutrient supplementation with BA, B2, DHA, D3 and PC alone and the combination thereof were assessed in neuronal cultures by comparison to the vehicle control. The concentrations of BA, B2, DHA, D3 and PC were respectively 200 μM, 0.25 μM, 2.5 μM, 0.25 μM and 20 μM.

Several conditions were studied and PC12 cells were supplemented with:
(1) BA only
(2) B2 only
(3) DHA only
(4) D3 only
(5) PC only

$$DHA + B 2 + D 3 \qquad ( 6 )$$

$$BA + DHA + B 2 + D 3 \qquad ( 7 )$$

$$PC + DHA + B 2 + D 3 \qquad ( 8 )$$

$$BA + PC + DHA + B 2 + D 3 \qquad ( 9 )$$

Supplementation of cells was performed in 6-fold in 96 wells plates. These conditions were compared to non-supplemented cells.

### Results

Image analysis of the neurite outgrowth showed that supplementation of BA, DHA and PC alone increased neurite outgrowth more than supplementation of vitamin B2 and D3 alone as compared to control in an in vitro PC12 neuronal cell model. Neurite outgrowth is expressed as percentage relative to control (100%). The findings are depicted in FIG. 8.

Surprisingly, the effect on neurite outgrowth of combined supplementation of the nutrients resulted in a larger effect than the sum of the individual effects of said nutrients. DHA combined with B2 and D3 (condition (6)) resulted in a synergistic effect on neurite outgrowth (mean calculated or expected neurite outgrowth was 132.4, the observed mean neurite outgrowth was 138.3). An even further synergistic effect was observed when the latter nutrients were combined with BA (condition (7); mean calculated or expected neurite outgrowth was 146.6, the observed mean neurite outgrowth was 149.2). A similar effect was observed when PC was supplemented together with DHA, B2 and D3 (condition (8); mean calculated or expected neurite outgrowth was 143, the observed mean neurite outgrowth was 152.8). Supplementation of PC12 cells with all five components together increased neurite outgrowth in a more than predicted manner based on the individual components, thereby indicating the presence of synergy between BA+DHA+PC+B2+D3 (condition (9); mean calculated or expected neurite outgrowth was 157.2, the observed mean neurite outgrowth was 167.2).

Overall, the results show that the combination of DHA, B2 and D3 synergistically increased neurite outgrowth and the same applies to when PC and/or BA were added to the combination.

### Example 6—Synergy Between B2, D3, BA and PC with Reference Diet

Reference is made to example 1 for PC12 neuronal cell cultures and measurement of neurite outgrowth with immunohistochemistry and imaging.

### Nutrient Supplementation

The effects of nutrient supplementation with vitamin B2, vitamin D3, PC and BA alone and the combination thereof with a reference diet were assessed in neuronal cultures by comparison to the vehicle control. The concentrations of B2, D3, PC and BA were respectively 0.25 μM, 0.75 μM, 15 μM, and 40 μM on the first plate. On a second plate, effects of supplementation with B2 (0.25 μM), D3 (0.75 μM) and BA (200 μM) and the combination thereof with a reference diet were assessed. The reference diet on both plates comprised the following ingredients: PC (5 μM), DHA (2 μM), choline-chloride (4 μM), vitamin C (15 μM), vitamin E (4 μM), vitamin B6 (2 μM), vitamin B9 (3 μM), vitamin B12 (0.02 μM) and selenium (0.016 μM). The mol % ratio of choline-chloride:PC in the reference diet was around 45:55. The combination of the reference diet with the supplementation of B2, D3. PC and BA on the first plate thus resulted in a total amount of 20 μM of PC and the mol % ratio of choline-chloride:PC was around 17:83.

Supplementation of cells was performed in 6-fold in 96 wells plates. These conditions were compared to non-supplemented cells.

### Results

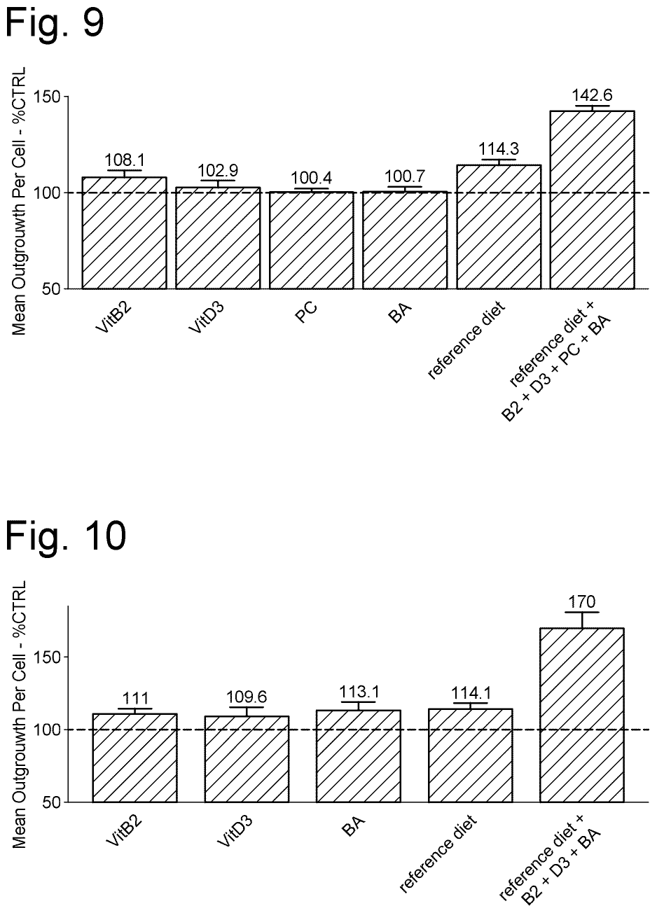

Image analysis of the neurite outgrowth showed that supplementation of B2, D3, PC (15 μM) and BA (40 μM) increased neurite outgrowth compared to control in an in vitro PC12 neuronal cell model. Neurite outgrowth is expressed as percentage relative to control (100%; dotted line). In addition, a synergistic effect was present based on the supplementation of these components together with the reference diet (mean calculated or expected neurite outgrowth was 126.4, the observed mean neurite outgrowth was 142.6). These findings are shown in FIG. 9.

Image analysis of the second plate showed a similar trend with an unexpected strong synergistic effect when nutrient supplementation of B2, D3, BA (200 μM) was combined with the reference diet (mean calculated or expected neurite outgrowth was 147.8, the observed mean neurite outgrowth was 170), see FIG. 10.

### Conclusion

The goal of these experiment was to see whether adding the nutrients of B2, D3, PC and BA would improve to outcome on neurite outgrowth compared to the reference diet which comprised ingredients all separately known for having beneficial effect of neuronal functioning and development. Surprisingly, the effect of the abovementioned components strongly increases the neurite outgrowth when given together with the reference diet more than would be expected based on their additional effect.

### Example 7—Functional Neurological Recovery after Acute Local Brain Injury in Rats

### Study Groups

Aged male Wistar rats (n=27; 14 months old) were kept in a 12-hour light-dark cycle with water and food ad libitum. All animals were fed the Control diet for 4 weeks prior to surgery. Animals were assigned to one of three groups comprised of 9 animals per group:
1) Brain injury and Control diet
2) Brain injury and Active diet
3) Sham injury and Control diet.

### Diets

Animals were given a Control diet and/or an Active diet for 51 days, which were isocaloric and contained the same amounts of macronutrients such as protein, carbohydrates and total fat. In addition, in both diets, 5 g/100 g diet cellulose was included as dietary fibre. Furthermore, the Active diet was similar to the Control diet, but was further enriched as outlined in Table 1 below for comparison.

TABLE 1
Nutrient supplementation in the Control diet versus the Active diet.

| | Control diet | Active diet |
|---|---|---|
| Ingredient | (per kg diet) | (per kg diet) |
| Fish oil (DHA/EPA) (g) | 0 | 40 |
| of which part is DHA (g) | 0 | 5.04 |
| of which part is EPA (g) | 0 | 7.84 |
| Choline chloride (g) | 2.3 | 8.295 |
| of which part is choline (g) | 0.998 | 3.600 |
| Lecithin (g) | 0 | 14.3369 |
| of which part is PC (g) | | 2.86738 |
| Vitamin A (IU) | 4000 | 16000 |
| Vitamin C (mg) | 0 | 1600 |
| Vitamin D3 (IU) | 1000 | 4000 |
| Vitamin E (IU) | 75 | 825 |
| Vitamin B1 (mg) | 5.0 | 20.0 |
| Vitamin B2 (mg) | 6.0 | 24.0 |
| Vitamin B6 (mg) | 6.0 | 24.0 |
| Vitamin B9 (mg) | 2.0 | 8.0 |
| Vitamin B12 (μg) | 25 | 100 |
| Magnesium (mg) | 507 | 1571 |
| Selenium (mg) | 0.17 | 0.77 |

Rats can synthesize their own vitamin C and thus the control diet did not contain any added vitamin C as part of their recommended daily amount. Furthermore, the mole % of choline in the active diet was 81% derived from choline-chloride and 19% derived from PC based on their molecular weight.

### Experimental Set-Up

Before surgery, animals were anaesthetised with 2-3% isoflurane in 95% 02 and transferred to a stereotaxic frame. As a control group, the sham injury consisted of skin incisions to expose the skull. Regarding the subjects in the study groups, skin incisions were made to expose the skull and four holes were drilled into the skull at the right side of the brain at the following co-ordinates in relation to bregma and midline:
a) AP+3.5 mm, ML 2.8 mm
b) AP+2 mm, ML 2.8 mm,
c) AP+0.5 mm, ML 2.8 mm and
d) AP-1 mm, ML 2.8 mm.

Local brain injuries were induced by injection of Endothelin-1 (Sigma-Aldrich, E7764) at each of the four sites, at a concentration of 0.25 μg/0.5 μL with a total volume of 0.5 μL using a microinjection pump (CMA/102 from CMA Microdialysis, AB Sweden) attached to a Hamilton syringe set to 0.2 μL/min. Injuries were mapped to the right sensorimotor cortical region, causing sustained impairment in somatosensory and motor function, allowing for longitudinal behavioural studies. After surgery with either sham injury or brain injury, the skin was sutured and rats were returned to their home cage.

Animals were kept for 51 days post-surgery and were subjected to different behavioural tests to assess loss and recovery of functional abilities. To assess global neurological impairment, the modified neurological severity score (mNSS) was performed at baseline and afterwards once a week for 7 weeks post-surgery. The mNSS is a composite score that takes motor, reflex, and balance function into account, see Table 2. Two animals were unable to complete the task at baseline and were excluded from the analysis. To assess skilled locomotion, the animals were subjected to a foot-slip test on a horizontal ladder at day 51 post-surgery. Animals were placed on the ladder which was raised 30 cm from the ground and foot misplacements were measured whenever a left hind paw extended below the grid as the rat traversed the ladder.

TABLE 2
Modified neurological severity scoring sheet

| Task | Score |
|---|---|
| Failure to exit 90 cm diameter hoop | |
| exit within 20 sec | 0 |
| exit within 40 sec | 1 |
| exit within 60 sec | 2 |
| Straight walk | 1 |
| Startle reflex | 1 |
| Pinna reflex | 1 |
| Corneal reflex | 1 |
| Seeking behaviour | 1 |
| Tail raised - hind flexion | 1 |
| Prostration | 1 |
| Righting reflex | 1 |
| Hemiplegia | 1 |
| Loss of paw placement (each paw) | 4 |
| Beam balance | |
| <60 sec | 1 |
| <40 sec | 2 |
| <20 sec | 3 |
| 5 cm beam walking | 1 |
| 3 cm beam walking | 1 |
| 2 cm beam walking | 1 |
| Maximum score | 21 |

### Results

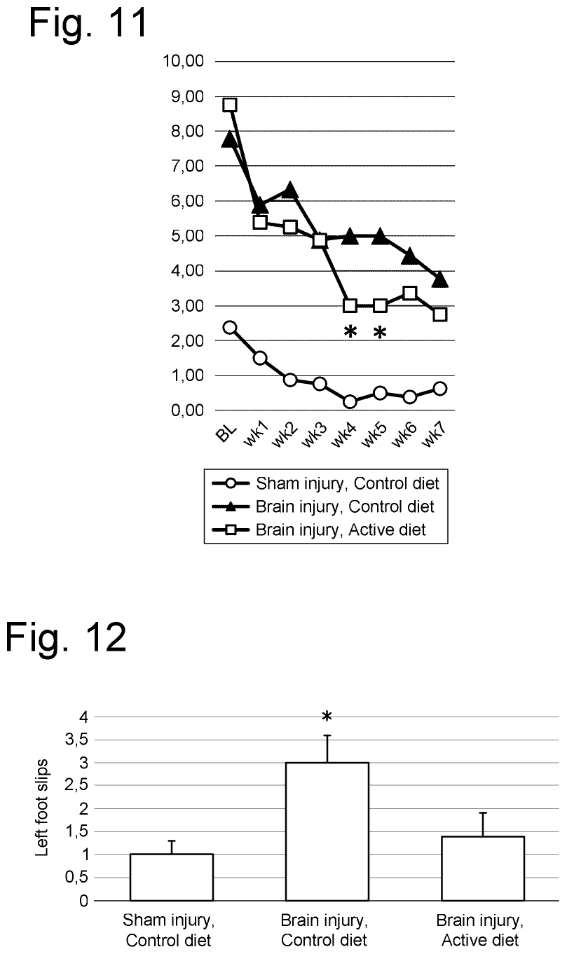

The mNSS results showed an overall effect of Treatment group (F(2,22)=103, p<0.001), Time (F(7,154)=42.27, p<0.001) and a significant Treatment by Time interaction (F(14,154)=4.75, p<0.001). This means that a there was a significant effect shown in the treatment group as the groups show differences in neurological impairment, that there was an effect over time in weeks as the mNSS score decreases over time and that there was an interaction between the study groups and the time in weeks as the change over time is different between the three study groups (see FIG. 11). Sidak's multiple comparison tests showed that animals subjected to brain injury and given the Active diet had significantly reduced neurological deficits at week 4 and week 5 post-injury when compared to the group subjected to brain injury but given the Control diet (see FIG. 11 at *).

Foot-slip test results showed a significant difference in left hind paw foot slips between Groups (F(2,18)=5.041, p<0.02). Tukey's post-hoc testing showed that animals subjected to brain injury and fed with the Control diet group made more foot-slips than the reference group (sham injury and fed with Control diet) and the group subjected to brain injury and fed with the Active diet at day 51 post-surgery, indicating that the active diet restored the impairment in skilled locomotion induced by the local brain injury (FIG. 12).

These results support that over time recovery from acute local brain injury is improved when given the nutrient supplementation in the Active diet. In particular, the Active diet supports functional recovery after brain damage, leading to a better and faster neurological recovery.

### Example 8—Tube Feed Formulation

An exemplary enteral tube feed was formulated comprising per 100 ml 128 kcal and comprising per 100 kcal 4.88 g protein, 4.5 g lipids and 9.3 g carbohydrates. The tube feed comprises ingredients according to table 1 and is for administration to a tube to a patient during recovery after having suffered neurotrauma or after being diagnosed with a neurodegenerative disease or during recovery after having suffered brain injury.

| Unit | Nutrient | Per 100 kcal |
|---|---|---|
| μg | Vitamin D3 | 2.08 |
| g | DHA | 0.04 |
| g | EPA | 0.06 |
| mg | Choline | 36.67 |
| mg | Short-chain GOS | 887 |
| mg | Long-chain FOS | 97.5 |
| mg | Low-viscosity pectin | 195 |
| mg | Magnesium | 24.7 |
| μg | Selenium | 7.5 |
| μg RE | Vitamin A | 85.71 |
| mg α-TE | Vitamin E | 2.32 |
| mg | Thiamin | 0.16 |
| mg | Riboflavin | 0.24 |
| mg NE | Niacin (equivalent) | 1.29 |
| mg | Pantothenic acid | 0.42 |
| mg | Vitamin B6 | 0.25 |
| μg | Folic acid | 24.24 |
| μg | Vitamin B12 | 0.60 |
| μg | Biotin | 2.91 |
| mg | Vitamin C | 18.17 |
| g | Phospholipids | 0.20 |

### Example 9—Oral Nutritional Supplement

An exemplary oral nutritional supplement was formulated comprising per 100 ml 240 kcal and comprising per 100 kcal 6 g protein, 3.89 g lipids and 9.57 g carbohydrates. The nutritional supplement comprises ingredients according to table 2 and is for administration as a drink to patients during recovery after having suffered neurotrauma or after being diagnosed with a neurodegenerative disease or during recovery after having suffered brain injury.

| Unit | Nutrient | Per 100 kcal |
|---|---|---|
| μg | Vitamin D3 | 3.33 |
| g | DHA | 0.08 |
| g | EPA | 0.12 |
| mg | Choline | 33.33 |
| mg | Short-chain GOS | 937 |
| mg | Long-chain FOS | 104 |
| mg | Low-viscosity pectin | 208 |
| mg | Magnesium | 58.3 |
| μg | Selenium | 13 |
| μg RE | Vitamin A | 125 |
| mg α-TE | Vitamin E | 4.17 |
| mg | Thiamin | 0.3 |
| mg | Riboflavin | 0.4 |
| mg NE | Niacin (equivalent) | 1.29 |
| mg | Pantothenic acid | 0.42 |
| mg | Vitamin B6 | 0.30 |
| μg | Folic acid | 66.67 |
| μg | Vitamin B12 | 1 |
| μg | Biotin | 3.5 |
| mg | Vitamin C | 31.67 |
| g | Phospholipids | 0.33 |

1.-11. (canceled)

12. A nutritional composition comprising a combination, based on 100 g dry weight, of:
i) 480-680 mg DHA;
ii) 1.23-1.53 mg vitamin B2;
iii) 38-54 μg vitamin D3;
iv) 200-370 mg choline;
v) 9.0-11.3 g dietary fibres,
wherein choline is provided in the form of a choline salt and phosphatidylcholine.

13. The nutritional composition according to claim 12, wherein the nutritional composition further comprises 100-170 mg EPA based on 100 g dry weight of the composition.

14. The nutritional composition according to claim 12, wherein 1-5 wt % choline in the nutritional composition is provided in the form of phosphatidylcholine.

15. A liquid nutritional composition comprising the combination of
iv) DHA;
v) vitamin B2;
vi) vitamin D3;
and one or more from
vii) choline or a salt thereof and/or phosphatidylcholine; and
viii) dietary fibres and/or dietary butyrate.

16. Liquid nutritional composition according to claim 15, wherein the composition comprises per 100 kcal
i) 20 to 100 mg DHA;
ii) 0.4 to 0.6 mg vitamin B2;
iii) 1.3 to 4.5 μg vitamin D3;
and further comprises
iv) 100 to 1000 mg choline or a salt thereof and/or phosphatidylcholine;
and/or
v) 0.75 to 2.5 g dietary fibres and/or 0.015 to 0.25 g dietary butyrate.

17. A method for the treatment of neurodegenerative diseases and/or neurotrauma and/or cerebral palsy, the method comprising administering to a subject in need thereof a nutritional composition comprising a therapeutically effective combination of
i) omega-3 fatty acids, wherein the omega-3 fatty acids comprises at least DHA;
ii) vitamin B2; and
iii) vitamin D3,
and one or more selected from
iv) choline or a salt thereof and/or phosphatidylcholine; and
v) dietary fibres and/or dietary butyrate,
wherein vitamin D3 is supplemented in an amount of 5 to 80 μg per day.

18. The method according to claim 17, wherein at least 40 mol % choline is provided in the form of phosphatidylcholine.

19. The method according to claim 17, wherein the nutritional composition further comprises one or more components selected from vitamin A, C and E, phospholipids, selenium, magnesium and further B vitamins.

20. The method according to claim 17, wherein the neurodegenerative disease is selected from Alzheimer's diseases, Parkinson's disease, Huntington's disease, frontotemporal dementia and Lewy body dementia.

21. The method according to claim 17, wherein the treatment of a neurodegenerative disease involves i.e. comprises treating and/or preventing and/or delaying the symptoms associated with said neurodegenerative disease in the subject or wherein the prodromal phase of said neurodegenerative disease has been diagnosed in the subject.

22. The method according to claim 17, wherein the neurotrauma is selected from traumatic brain injury (TBI), spinal cord injury (SCI), and peripheral nerve injuries.

23. The method according to claim 22, wherein the neurotrauma is caused by external factors selected from one or more of:
occupational hazards including car, sports, and workplace accidents;
explosions and blast injuries;
electric shock;
blunt force trauma;
penetrating injuries involving the penetration of an external object, including a bullet or a sharp object, into the brain, spinal cord or peripheral nerves.

24. The method according to claim 17, wherein the treatment of neurodegenerative diseases involves, i.e. comprises an increase in neurite outgrowth in subjects suffering from a neurodegenerative disease and treated with the composition as compared to subjects suffering from said neurodegenerative disease and not treated with the composition and/or wherein the treatment of neurotrauma involves, i.e. comprises an increase in length of neuronal outgrowth in subjects suffering from neurotrauma and treated with the composition as compared to subjects suffering from said neurotrauma and not treated with the composition.

25. The method according to claim 17, wherein the composition is used daily for at least 4 weeks after diagnosis of said neurodegenerative disease and/or after diagnosis or occurrence of said neurotrauma.

26. The method according to claim 17, wherein the composition comprises daily dosages of:
i) 150 to 1200 mg DHA per day;
ii) 0.8 to 7.6 mg vitamin B2;
iii) 6 to 75 μg vitamin D3;
and one or more of:
iv) 200 to 1400 mg choline based on molar weight of choline, wherein the choline is selected from free choline, choline salts and/or choline esters;
and/or
v) 2.5 to 12.5 g dietary fibres and/or 0.125 g to 0.65 g dietary butyrate.

27. The method according to claim 17, wherein the nutritional composition is a liquid nutritional composition.

28. The method according to claim 21, wherein the symptoms involve, i.e comprises, one or more of:
cognitive decline associated with memory loss, reduced attention span, reduced executive function, impairment in performing activities of daily living; and/or
motor symptoms associated with tremors, rigidity, bradykinesia, coordination and balance; and/or
behavioural and psychological changes associated with depression, anxiety, irritability, fatigue, apathy, social withdrawal and mood swings; and/or
sensory disturbances associated with loss of sensation, numbness, tingling, or altered perception of touch, pain or temperature; and/or
sleep disturbances associated with insomnia, excessive daytime sleepiness, fragmented sleep, restless legs syndrome and rapid eye movement (REM) sleep behaviour disorder; and/or
autonomic dysfunction associated with orthostatic hypotension, urinary problems, incontinence, constipation, changes in sweating and body temperature regulation.
